(19) United States
(12) Patent Application Publication
(10) Pub. No.: US 2008/0091025 A1
(43) Pub. Date: Apr. 17, 2008

(54) AROMATIC AMINE DERIVATIVE AND ORGANIC ELECTROLUMINESCENCE DEVICE USING THE SAME

(75) Inventors: Hironobu MORISHITA, Chiba (JP); Nobuhiro Yabunouchi, Chiba (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Idemitsu Kosan Co., Ltd., Tokyo (JP)

(21) Appl. No.: 11/854,162

(22) Filed: Sep. 12, 2007

(30) Foreign Application Priority Data
Sep. 15, 2006 (JP) 2006-250568

(51) Int. Cl. C07C 211/54; C07D 263/62; C07D 277/62; C07D 417/10; H01J 1/62

(52) U.S. Cl. 548/156; 548/100; 548/152; 548/160; 548/219

(57) ABSTRACT

The present invention provides an organic electroluminescence device which can be driven at a reduced voltage, hardly causes the crystallization of a molecule, can be produced in improved yield, and has a long lifetime because of difficulty of molecular crystallization, and aromatic amine derivatives for realizing the device. The aromatic amine derivatives are novel aromatic amine derivatives having a specific structure. The organic electroluminescence device includes an organic thin film layer formed of one or more layers including at least a light emitting layer, the organic thin film layer being interposed between a cathode and an anode. In the organic electroluminescence device, at least one layer of the organic thin film layer, especially a hole transporting layer, contains the aromatic amine derivative alone or as a component of a mixture.

### TECHNICAL FIELD

The present invention relates to an aromatic amine derivative and an organic electroluminescence (EL) device using the same, in particular, to aromatic amine derivative realizing the organic EL device capable of suppressing the crystallization of a molecule while decreasing a driving voltage, improving yields upon production of the organic EL device, and of increasing the lifetime of the organic EL device by using the aromatic amine derivative having a specific substituent as a hole transporting material.

### BACKGROUND ART

An organic EL device is a spontaneous light emitting device which utilizes the principle that a fluorescent substance emits light by energy of recombination of holes injected from an anode and electrons injected from a cathode when an electric field is applied. Since an organic EL device of the laminate type driven under low electric voltage was reported by C. W. Tang et al. of Eastman Kodak Company (C. W. Tang and S. A. Vanslyke, Applied Physics Letters, Volume 51, Pages 913, 1987 or the like), many studies have been conducted on organic EL devices using organic materials as the constituent materials. Tang et al. used tris(8-quinolinolato)aluminum for a light emitting layer and a triphenyldiamine derivative for a hole transporting layer. Advantages of the laminate structure are that the efficiency of hole injection into the light emitting layer can be increased, that the efficiency of forming exciton which are formed by blocking and recombining electrons injected from the cathode can be increased, and that exciton formed within the light emitting layer can be enclosed. As described above, for the structure of the organic EL device, a two-layered structure having a hole transporting (injecting) layer and an electron-transporting light emitting layer and a three-layered structure having a hole transporting (injecting) layer, a light emitting layer, and an electron-transporting (injecting) layer are well known. To increase the efficiency of recombination of injected holes and electrons in the devices of the laminate type, the structure of the device and the process for forming the device have been studied.

In general, when an organic EL device is driven or stored in an environment of high temperature, adverse effects such as a change in the luminescent color, a decrease in emission efficiency, an increase in the driving voltage, and a decrease in the lifetime of light emission arise. To prevent the adverse effects, it has been necessary that the glass transition temperature (Tg) of the hole transporting material be elevated. Therefore, it is necessary that the many aromatic groups be held within the molecule of the hole transporting material, for example, the aromatic diamine derivative in Patent Document 1 and the fused aromatic ring diamine derivative in Patent Document 2, and in general, a structure having 8 to 12 benzene rings may preferably be used.

However, when a large number of aromatic groups are present in a molecule, crystallization is apt to occur upon production of an organic EL device through the formation of a thin film by using those hole transporting materials. As a result, there arises a problem such as the clogging of the outlet of a crucible to be used in vapor deposition or a reduction in yields of the organic EL device due to the generation of a fault of the thin film resulting from the crystallization. In addition, a compound having a large number of aromatic groups in any one of its molecules generally has a high glass transition temperature (Tg), but has a high sublimation temperature. Accordingly, there arises a problem in that the lifetime is short because a phenomenon such as decomposition at the time of vapor deposition or the formation of a nonuniform deposition film is expected to occur.

Meanwhile, there is a known document disclosing an asymmetric aromatic amine derivative. For example, Patent Document 3 describes an aromatic amine derivative having an asymmetric structure. However, the document has no specific example, and has no description concerning characteristics of an asymmetric compound. In addition, Patent Document 4 describes an asymmetric aromatic amine derivative having phenanthrene as an example. However, the derivative is treated in the same way as that of a symmetric compound, and the document has no description concerning characteristics of an asymmetric compound. In addition, none of those patents explicitly describes a method of producing an asymmetric compound in spite of the fact that the asymmetric compound requires a special synthesis method. Further, Patent Document 5 describes a method of producing an aromatic amine derivative having an asymmetric structure, but has no description concerning characteristics of an asymmetric compound. Patent Document 6 describes an asymmetric compound which has a high glass transition temperature and which is thermally stable, but exemplifies only a compound having carbazole.

In addition, Patent Document 7 reports an organic EL material introducing benzobisthiadiazole as its central skeleton; provided that Patent Document 7 reports only an example in which the material is applied to the light emitting layer of an organic EL device, and has no description concerning the performance of the material when the material is used in a hole transporting layer. Further, the material uses benzobisthiadiazole as its central skeleton, so the following problem and concern arise: the material is apt to crystallize, and the characteristics (such as an ionization potential, a carrier mobility, and electrical or thermal durability) of the material may be largely different from those requested of a material for a hole transporting (injecting) layer.

As described above, an organic EL device having a long lifetime has been reported, but it cannot be said yet that the device always shows sufficient performance. In view of the foregoing, the development of an organic EL device having further excellent performance has been strongly desired.

[Patent Document 1] U.S. Pat. No. 4,720,432

[Patent Document 2] U.S. Pat. No. 5,061,569

[Patent Document 3] JP-A-08-48656

[Patent Document 4] JP-A-11-135261

[Patent Document 5] JP-A-2003-171366

[Patent Document 6] U.S. Pat. No. 6,242,115

[Patent Document 7] JP-A-10-340786

### DISCLOSURE OF THE INVENTION

### Problems to be Solved by the Invention

The present invention has been made with a view to solving the above-mentioned problems, and an object of the present invention is to provide an organic EL device in which a driving voltage is decreased and a molecule hardly crystallizes, which can be produced with improved yields, and which has a long lifetime, and aromatic amine derivatives realizing the organic EL device.

### Means for Solving the Problems

The inventors of the present invention have made extensive studies with a view toward achieving the above-mentioned object. As a result, they have found that the above-mentioned problems can be solved by using a novel aromatic amine derivative having a specific substituent represented by the following general formula (1) as a material for an organic EL device, in particular, a hole transporting material, thereby completing the present invention.

In addition, the inventors have found that an amino group substituted by an aryl group having a thiophene structure represented by a general formula (2) is suitable as an amine unit having a specific substituent. The inventors have found the following. That is, the amine unit has a polar group, and hence can interact with an electrode, whereby the injection of charge is facilitated, and the facilitation has a reducing effect on the driving voltage. In addition, the unit has steric hindrance, and hence an interaction between the molecules of the material is small, whereby the following effect is obtained: the crystallization of the material is suppressed, the yield in which an organic EL device is produced is improved, and the lifetime of an organic EL device to be obtained is lengthened. In particular, the combination of the material with a blue light emitting device exerts a significant reducing effect on the driving voltage and a significant lengthening effect on the lifetime of the device. Further, a compound having an asymmetric structure out of the compounds each having a large molecular weight can be deposited from the vapor at a lower temperature, so the decomposition of the compound at the time of deposition can be suppressed, and the lifetime can be lengthened.

The present invention provides an aromatic amine derivative represented by the following general formula (1):

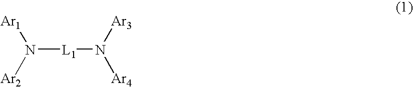

where:

L₁ represents a substituted or unsubstituted arylene group having 5 to 50 ring carbon atoms, or a substituted or unsubstituted heteroarylene group having 5 to 50 ring carbon atoms;

at least one of Ar₁ to Ar₄ is represented by the following general formula (2)

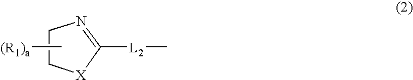

where

R₁ represents a hydrogen atom, a substituted or unsubstituted aryl group having 5 to 50 ring carbon atoms, a substituted or unsubstituted alkyl group having 1 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, an amino group substituted by a substituted or unsubstituted aryl group having 5 to 50 ring carbon atoms, a halogen atom, a cyano group, a nitro group, a hydroxy group, or a carboxyl group,

a represents an integer of 0 to 2,

X represents a sulfur atom, an oxygen atom, a selenium atom, or a tellurium atom,

L₂ represents a substituted or unsubstituted arylene group having 5 to 50 ring carbon atoms, or a substituted or unsubstituted heteroarylene group having 5 to 50 ring carbon atoms, and

multiple R₁ₛ may be bonded to each other to form a saturated or unsaturated, five- or six-membered cyclic structure which may be substituted; and

remaining groups of Ar₁ to Ar₄ which is not represented by the general formula (2) each independently represent a substituted or unsubstituted aryl group having 5 to 50 ring carbon atoms, or a substituted or unsubstituted heteroaryl group having 5 to 50 ring carbon atoms.

Further, the present invention provides an organic EL device including one or multiple organic thin film layers including at least a light emitting layer, the one or multiple organic thin film layers being interposed between a cathode and an anode, in which at least one layer of the one or more multiple organic thin film layers contains the aromatic amine derivative alone or as a component of a mixture.

### EFFECT OF THE INVENTION

An organic EL device using the aromatic amine derivative of the present invention hardly causes the crystallization of a molecule while decreasing a driving voltage, can be produced with improved yields, and has a long lifetime.

### BEST MODE FOR CARRYING OUT THE INVENTION

An aromatic amine derivative of the present invention is represented by the following general formula (1).

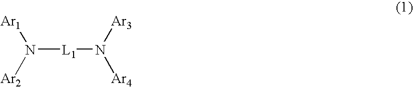

In the general formula (1):

L₁ represents a substituted or unsubstituted arylene group having 5 to 50 ring carbon atoms, or a substituted or unsubstituted heteroarylene group having 5 to 50 ring carbon atoms; and at least one of Ar₁ to Ar₄ is represented by the following general formula (2)

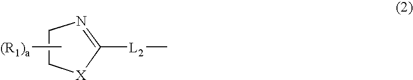

In the general formula (2),

R₁ represents a hydrogen atom, a substituted or unsubstituted aryl group having 5 to 50 ring carbon atoms, a substituted or unsubstituted alkyl group having 1 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, an amino group substituted by a substituted or unsubstituted aryl group having 5 to 50 ring carbon atoms, a halogen atom, a cyano group, a nitro group, a hydroxy group, or a carboxyl group, a represents an integer of 0 to 2, X represents a sulfur atom, an oxygen atom, a selenium atom, or a tellurium atom, L₂ represents a substituted or unsubstituted arylene group having 5 to 50 ring carbon atoms, or a substituted or unsubstituted heteroarylene group having 5 to 50 ring carbon atoms, and multiple R¹s may be bonded to each other to form a saturated or unsaturated, five- or six-membered cyclic structure which may be substituted.

In the general formula (1), remaining groups of Ar₁ to Ar₄ none of which is represented by the general formula (2) each independently represent a substituted or unsubstituted aryl group having 5 to 50 ring carbon atoms, or a substituted or unsubstituted heteroaryl group having 5 to 50 ring carbon atoms.

In the aromatic amine derivative of the present invention, Ar₁ in the general formula (1) is preferably represented by the general formula (2).

In the aromatic amine derivative of the present invention, Ar₁ and Ar₂ in the general formula (1) are preferably each represented by the general formula (2).

In the aromatic amine derivative of the present invention, Ar₁ and Ar₃ in the general formula (1) are preferably each represented by the general formula (2).

In the aromatic amine derivative of the present invention, three or more of Ar₁ to Ar₄ in the general formula (1) are preferably different from and asymmetric with respect to one another.

In the aromatic amine derivative of the present invention, three of Ar₁ to Ar₄ in the general formula (1) are preferably identical to and asymmetric with respect to one another.

In the aromatic amine derivative of the present invention, L₁ in the general formula (1) preferably represents a biphenylene group, a terphenylene group, or a fluorenylene group.

In the aromatic amine derivative of the present invention, L₂ in the general formula (2) preferably represents a phenylene group or a naphthylene group.

In the aromatic amine derivative of the present invention, at least one of Ar₁ to Ar₄ in the general formula (1) is preferably represented by the following general formula (3):

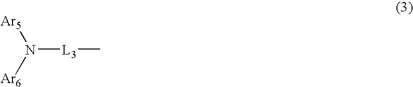

In the general formula (3):

Ar₅ and Ar₆ each independently represent a substituted or unsubstituted aryl group having 5 to 50 ring carbon atoms, a substituted or unsubstituted heteroaryl group having 5 to 50 ring carbon atoms, or a substituent represented by the general formula (2); and L₃ represents a substituted or unsubstituted arylene group having 5 to 50 ring carbon atoms, or a substituted or unsubstituted heteroarylene group having 5 to 50 ring carbon atoms.

In the aromatic amine derivative of the present invention, Ar₂ in the general formula (1) is preferably represented by the general formula (3).

In the aromatic amine derivative of the present invention, Ar₂ and Ar₄ in the general formula (1) are preferably each independently represented by the general formula (3).

In the aromatic amine derivative of the present invention, X in the general formula (2) preferably represents a sulfur atom.

Examples of the substituted or unsubstituted aryl group having 5 to 50 ring carbon atoms and substituted or unsubstituted heteroaryl group having 5 to 50 as ring carbon atoms each represented by any one of Ar₁ to Ar₄ in the general formulae (1), R₁ in the general formula (2), and Ar₅ and Ar₆ in the general formula (3) include a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl-4-yl group, a p-terphenyl-3-yl group, a p-terphenyl-2-yl group, an m-terphenyl-4-yl group, an m-terphenyl-3-yl group, an m-terphenyl-2-yl group, an o-tolyl group, an m-tolyl group, a p-tolyl group, a p-t-butylphenyl group, a p-(2-phenylpropyl)phenyl group, a 3-methyl-2-naphthyl group, a 4-methyl-1-naphthyl group, a 4-methyl-1-anthryl group, a 4′-methylbiphenylyl group, a 4″-t-butyl-p-terphenyl-4-yl group, a fluoranthenyl group, a fluorenyl group, a 1-pyrrolyl group, a 2-pyrrolyl group, a 3-pyrrolyl group, a pyradinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a quinolyl group, a 3-quinolyl group, a 4-quinolyl group, a 5-quinolyl group, a 6-quinolyl group, a 7-quinolyl group, an 8-quinolyl group, a 1-isoquinolyl group, a 3-isoquinolyl group, a 4-isoquinolyl group, a 5-isoquinolyl group, a 6-isoquinolyl group, a 7-isoquinolyl group, an 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, a 1-phenanthridinyl group, a 2-phenanthridinyl group, a 3-phenanthridinyl group, a 4-phenanthridinyl group, a 6-phenanthridinyl group, a 7-phenanthridinyl group, an 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthrolin-2-yl group, a 1,7-phenanthrolin-3-yl group, a 1,7-phenanthrolin-4-yl group, a 1,7-phenanthrolin-5-yl group, a 1,7-phenanthrolin-6-yl group, a 1,7-phenanthrolin-8-yl group, a 1,7-phenanthrolin-9-yl group, a 1,7-phenanthrolin-10-yl group, a 1,8-phenanthrolin-2-yl group, a 1,8-phenanthrolin-3-yl group, a 1,8-phenanthrolin-4-yl group, a 1,8-phenanthrolin-5-yl group, a 1,8-phenanthrolin-6-yl group, a 1,8-phenanthrolin-7-yl group, a 1,8-phenanthrolin-9-yl group, a 1,8-phenanthrolin-10-yl group, a 1,9-phenanthrolin-2-yl group, a 1,9-phenanthrolin-3-yl group, a 1,9-phenanthrolin-4-yl group, a 1,9-phenanthrolin-5-yl group, a 1,9-phenanthrolin-6-yl group, a 1,9-phenanthrolin-7-yl group, a 1,9-phenanthrolin-8-yl group, a 1,9-phenanthrolin-10-yl group, a 1,10-phenanthrolin-2-yl group, a 1,10-phenanthrolin-3-yl group, a 1,10-phenanthrolin-4-yl group, a 1,10-phenanthrolin-5-yl group, a 2,9-phenanthrolin-1-yl group, a 2,9-phenanthrolin-3-yl group, a 2,9-phenanthrolin-4-yl group, a 2,9-phenanthrolin-5-yl group, a 2,9-phenanthrolin-6-yl group, a 2,9-phenanthrolin-7-yl group, a 2,9-phenanthrolin-8-yl group, a 2,9-phenanthrolin-10-yl group, a 2,8-phenanthrolin-1-yl group, a 2,8-phenanthrolin-3-yl group, a 2,8-phenanthrolin-4-yl group, a 2,8-phenanthrolin-5-yl group, a 2,8-phenanthrolin-6-yl group, a 2,8-phenanthrolin-7-yl group, a 2,8-phenanthrolin-9-yl group, a 2,8-phenanthrolin-10-yl group, a 2,7-phenanthrolin-1-yl group, a 2,7-phenanthrolin-3-yl group, a 2,7-phenanthrolin-4-yl group, a 2,7-phenanthrolin-5-yl group, a 2,7-phenanthrolin-6-yl group, a 2,7-phenanthrolin-8-yl group, a 2,7-phenanthrolin-9-yl group, a 2,7-phenanthrolin-10-yl group, a 1-phenadinyl group, a 2-phenadinyl group, a 1-phenothiadinyl group, a 2-phenothiadinyl group, a 3-phenothiadinyl group, a 4-phenothiadinyl group, a 10-phenothiadinyl group, a 1-phenoxadinyl group, a 2-phenoxadinyl group, a 3-phenoxadinyl group, a 4-phenoxadinyl group, a 10-phenoxadinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrol-1-yl group, a 2-methylpyrrol-3-yl group, a 2-methylpyrrol-4-yl group, a 2-methylpyrrol-5-yl group, a 3-methylpyrrol-1-yl group, a 3-methylpyrrol-2-yl group, a 3-methylpyrrol-4-yl group, a 3-methylpyrrol-5-yl group, a 2-t-butylpyrrol-4-yl group, a 3-(2-phenylpropyl)pyrrol-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, and a 4-t-butyl-3-indolyl group. Of those, a phenyl group, a naphthyl group, a biphenylyl group, a terphenylyl group, and a fluorenyl group are preferable.

Examples of the substituted or unsubstituted arylene group having 5 to 50 ring carbon atoms and the substituted or unsubstituted heteroarylene group having 5 to 50 ring carbon atoms each represented by any one of L₁ in the general formula (1), L₂ in the general formula (2), and L₃ in the general formula (3) include groups obtained by turning the examples of the aryl group and the heteroaryl group into divalent groups.

Examples of the substituted or unsubstituted alkyl group having 1 to 50 carbon atoms represented by R₁ in the general formula (2) include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyan o-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, and a 2-norbornyl group.

The substituted or unsubstituted alkoxy group having 1 to 50 carbon atoms as R₁ in the general formula (2) is represented by —OY, and examples of Y include the same examples as those described for the above-mentioned alkyl group.

Examples of the substituted or unsubstituted aralkyl group having 6 to 50 carbon atoms as R₁ in the general formula (2) include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, a 1-pyrrolylmethyl group, a 2-(1-pyrrolyl)ethyl group, a p-methylbenzyl group, an m-methylbenzyl group, an o-methylbenzyl group, a p-chlorobenzyl group, an m-chlorobenzyl group, an o-chlorobenzyl group, a p-bromobenzyl group, an m-bromobenzyl group, an o-bromobenzyl group, a p-iodobenzyl group, an m-iodobenzyl group, an o-iodobenzyl group, a p-hydroxybenzyl group, an m-hydroxybenzyl group, an o-hydroxybenzyl group, a p-aminobenzyl group, an m-aminobenzyl group, an o-aminobenzyl group, a p-nitrobenzyl group, an m-nitrobenzyl group, an o-nitrobenzyl group, a p-cyanobenzyl group, an m-cyanobenzyl group, an o-cyanobenzyl group, a 1-hydroxy-2-phenylisopropyl group, and a 1-chloro-2-phenylisopropyl group.

The substituted or unsubstituted aryloxy group having 5 to 50 ring carbon atoms as R₁ in the general formula (2) is represented by —OY′, and examples of Y′ include examples similar to those described for the aryl group.

The substituted or unsubstituted arylthio group having 5 to 50 ring carbon atoms as R₁ in the general formula (2) is represented by —SY′, and examples of Y′ include examples similar to those described for the aryl group.

The substituted or unsubstituted alkoxycarbonyl group having 2 to 50 carbon atoms as R₁ in the general formula (2) is a group represented by —COOY, and examples of Y include examples similar to those described for the alkyl group.

Examples of a substituted or unsubstituted aryl group having 5 to 50 ring carbon atoms in the amino group substituted by the aryl group as R₁ in the general formula (2) include examples similar to those described for the aryl group.

Examples of the halogen atom as R₁ in the general formula (2) include a fluorine atom, a chlorine atom, a bromine atom, and an iodine atom.

In the general formula (2), a represents an integer of 0 to 2, and, when a represents 2, multiple R₁'s may be bonded to each other to form a saturated or unsaturated, five-membered or six-membered cyclic structure which may be substituted.

Examples of the five-membered or six-membered cyclic structure which may be formed include: cycloalkanes each having 4 to 12 carbon atoms such as cyclopentane, cyclohexane, adamantane, and norbornane; cycloalkenes each having 4 to 12 carbon atoms such as cyclopentene and cyclohexene; cycloalkadienes each having 6 to 12 carbon atoms such as cyclopentadiene and cyclohexadiene; and aromatic rings each having 6 to 50 carbon atoms such as benzene, naphthalene, phenanthrene, anthracene, pyrene, chrysene, and acenaphthylene.

Specific examples of the aromatic amine derivative represented by the general formula (1) of the present invention are shown below. However, the present invention is not limited to these exemplified compounds.

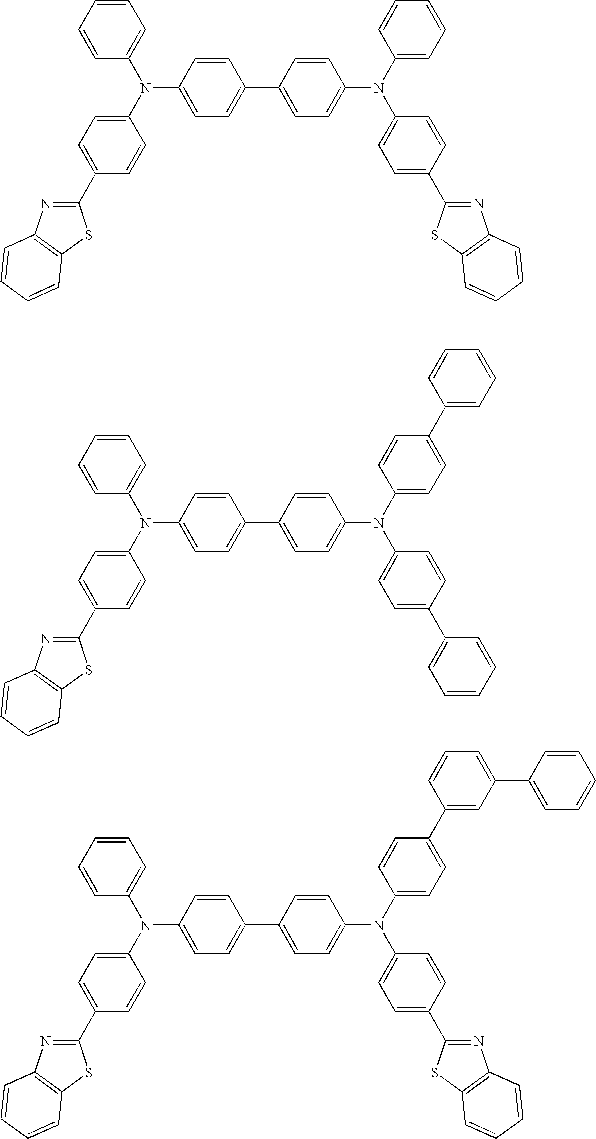

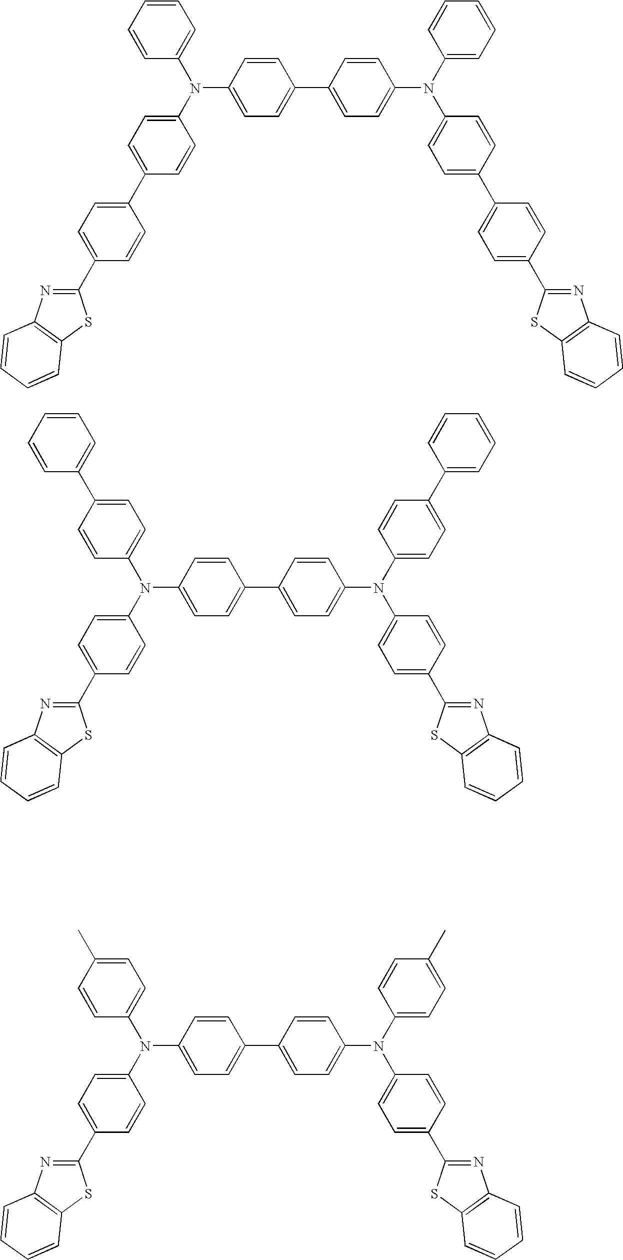

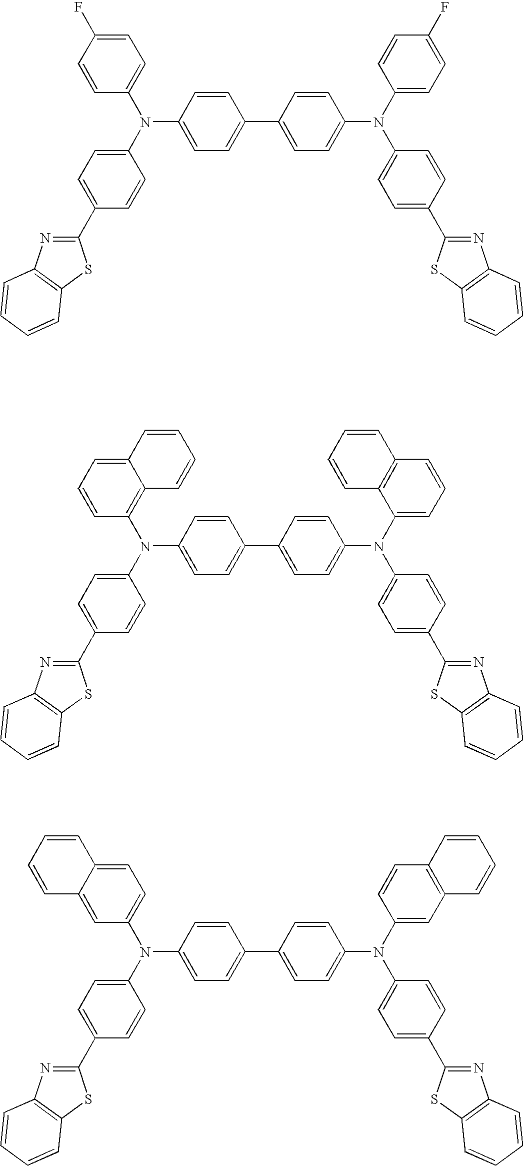

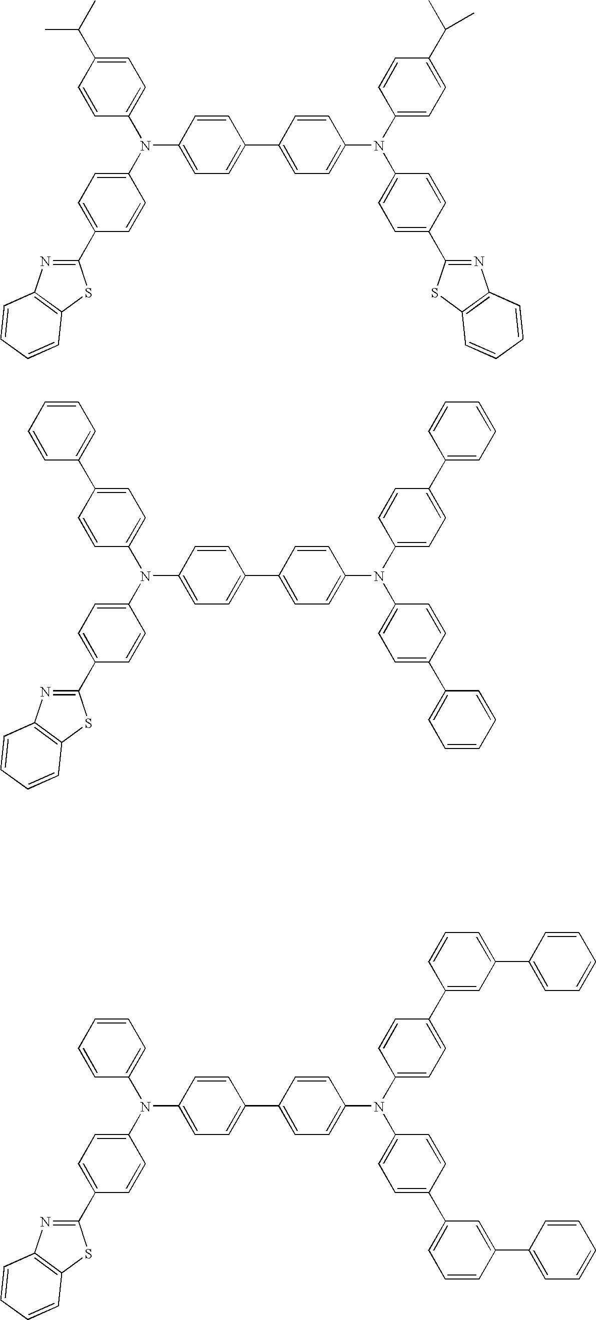

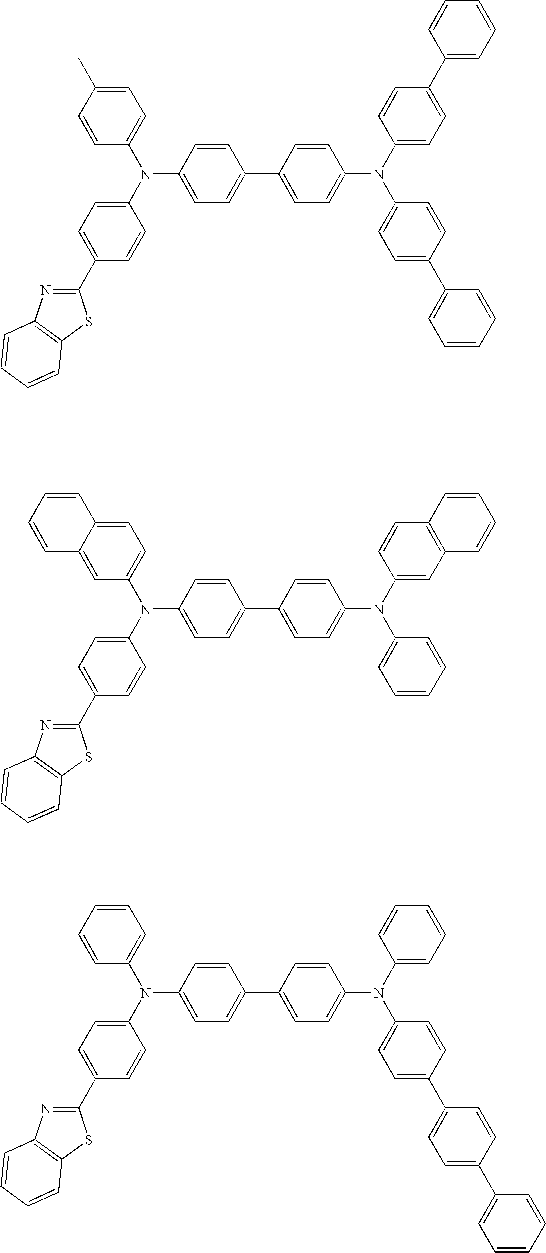

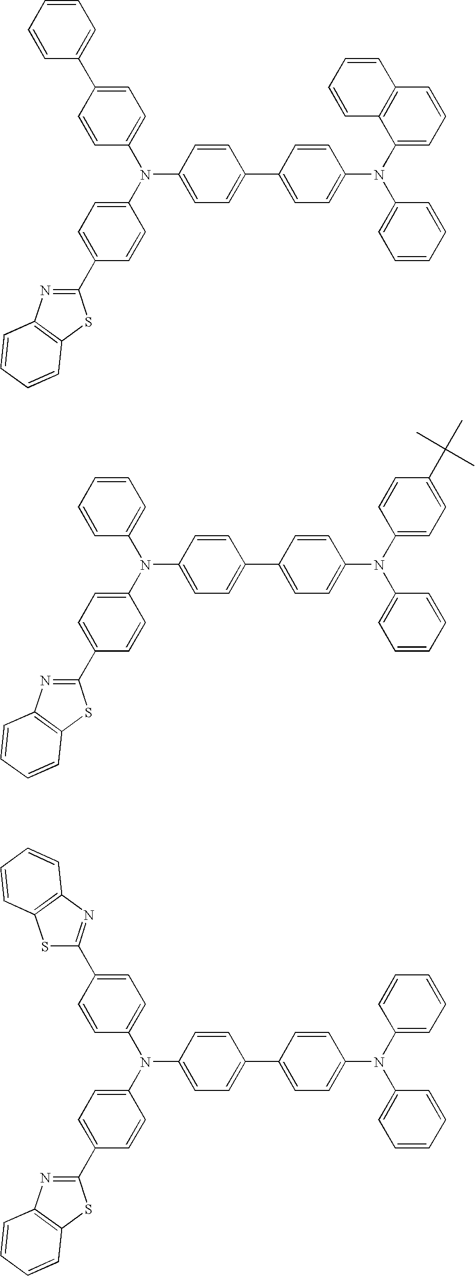

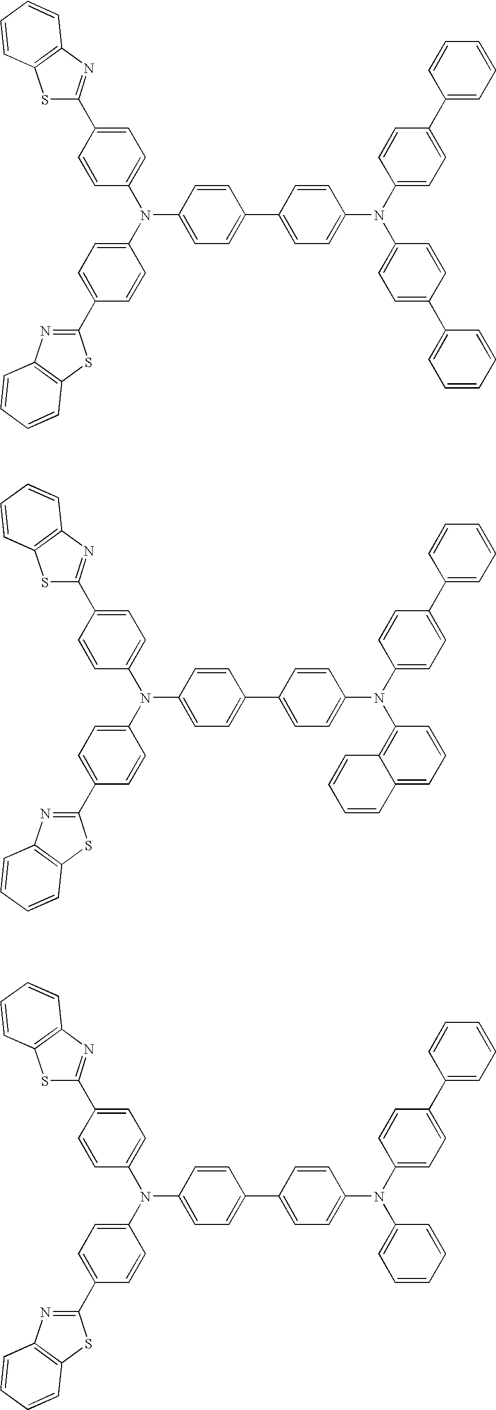

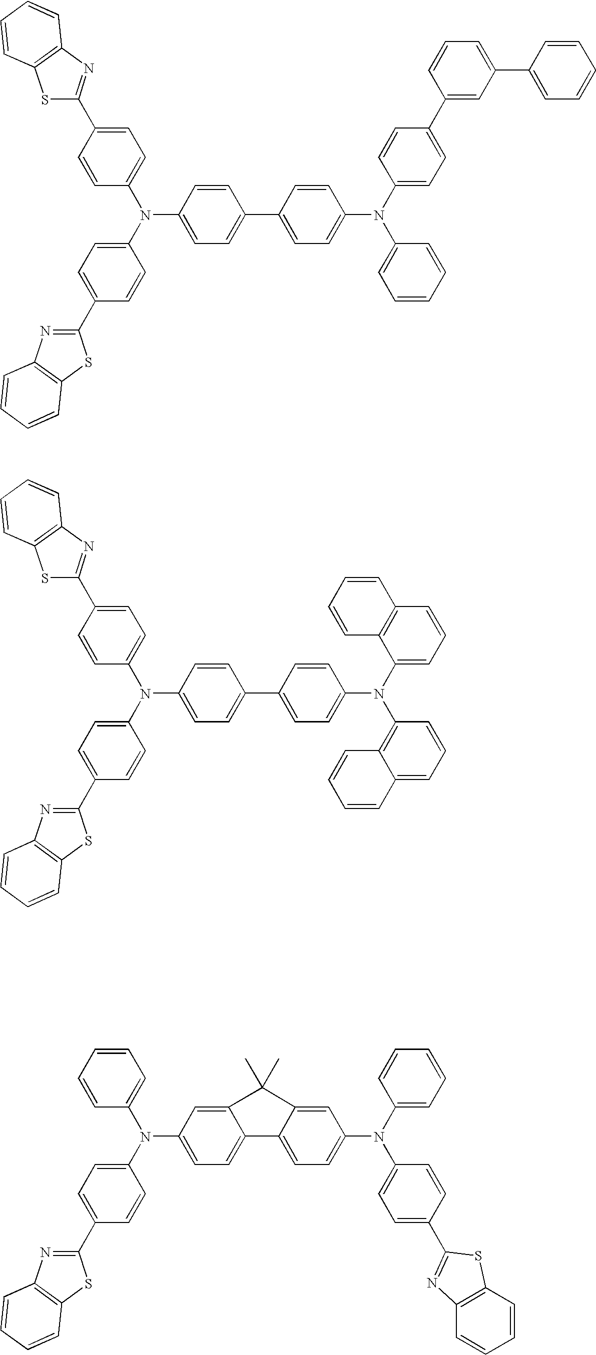

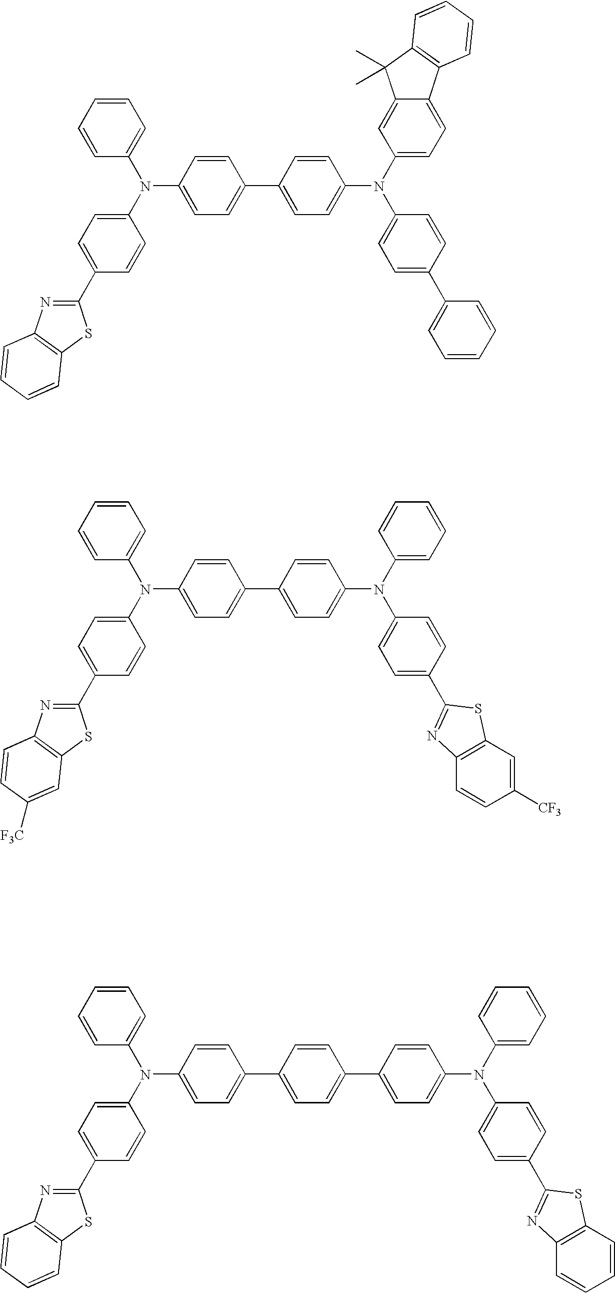

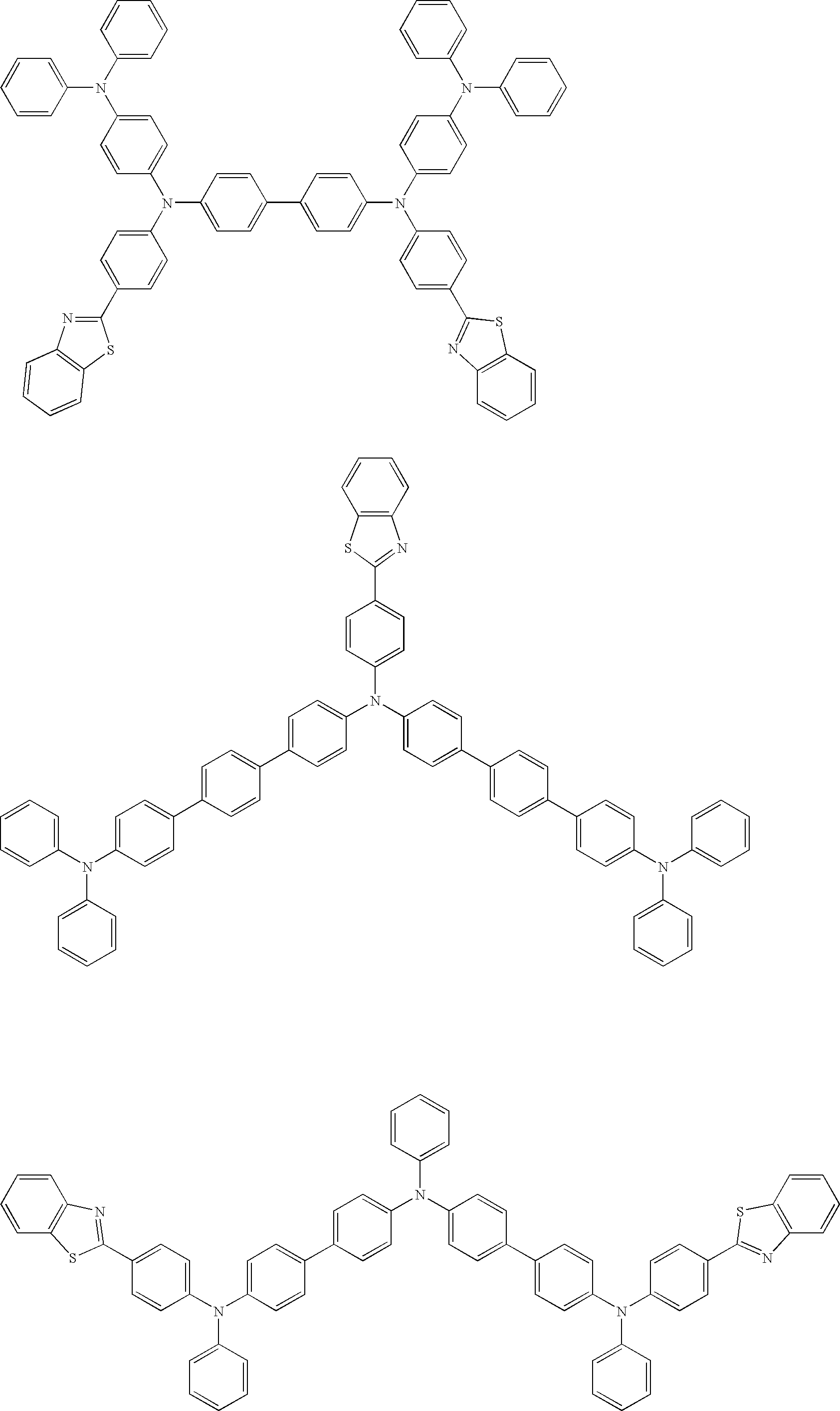

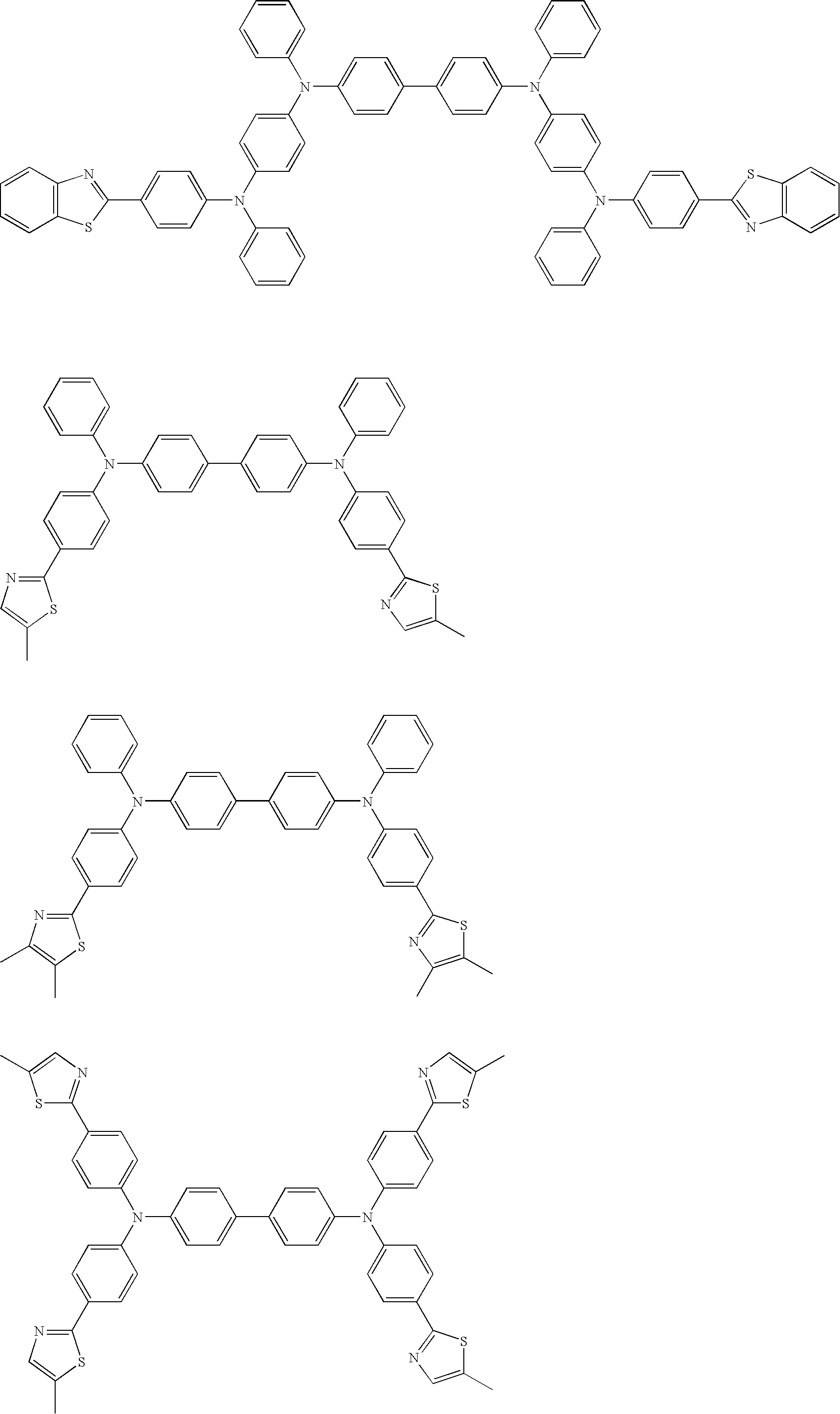

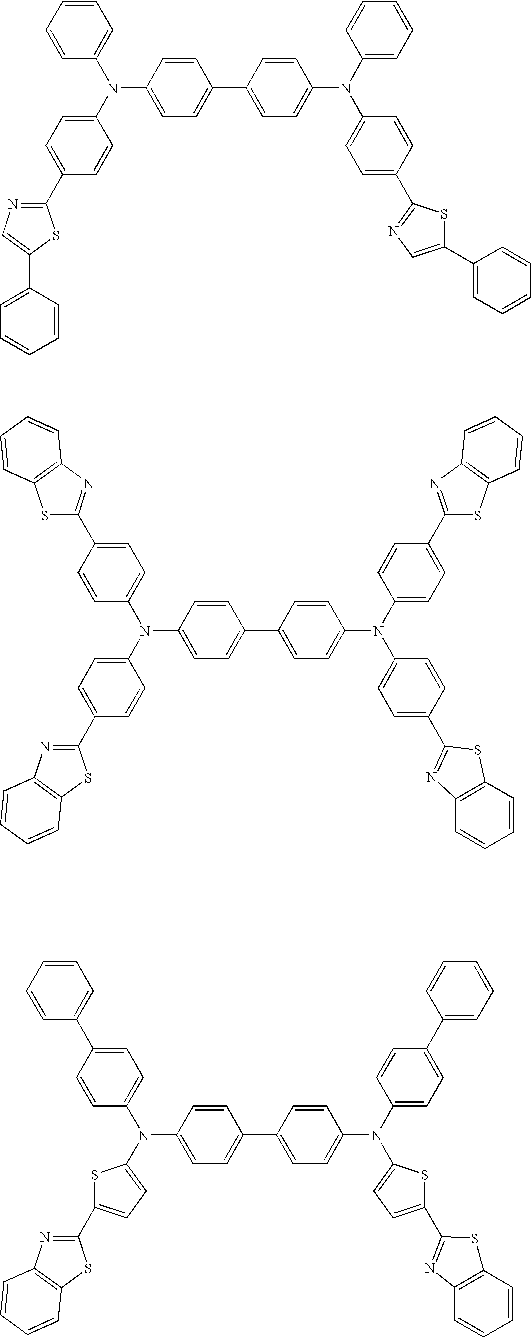

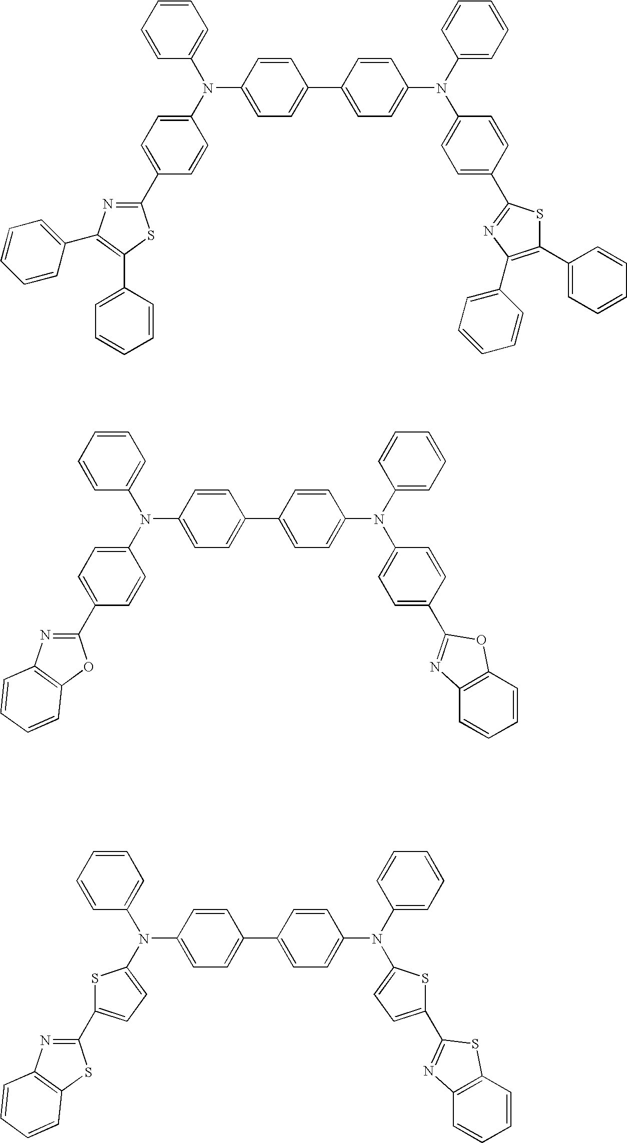

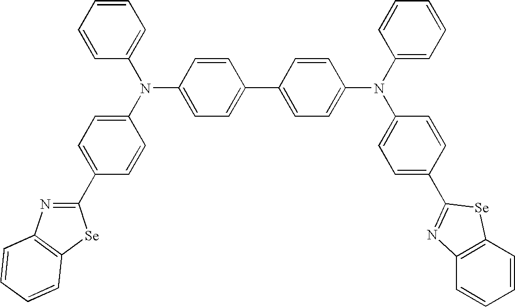

The aromatic amine derivative of the present invention is preferably a material for an organic electroluminescence device.

The aromatic amine derivative of the present invention is preferably a hole transporting material for an organic electroluminescence device.

An organic EL device of the present invention is preferably an organic EL device including one or multiple organic thin film layers including at least a light emitting layer, the one or multiple organic thin film layers being interposed between a cathode and an anode, in which at least one layer of the one or more multiple organic thin film layers contains the aromatic amine derivative alone or as a component of a mixture.

The organic EL device of the present invention, which can be used in a light emitting zone or a hole transporting zone, is preferably incorporated into the hole transporting zone.

The organic EL device of the present invention is preferably such that the organic thin film layer has a hole transporting layer, and the aromatic amine derivative is incorporated into the hole transporting layer.

The organic EL device of the present invention is preferably such that the organic thin film layer has a hole injecting layer, and the aromatic amine derivative is incorporated into the hole injecting layer. Further, the aromatic amine derivative is preferably incorporated as a main component into the hole injecting layer.

The aromatic amine derivative of the present invention is particularly preferably used in an organic EL device that emits bluish light.

The structure of the organic EL device of the present invention will be described in the following.

(1) Organic EL Device Structure

Typical examples of the structure of the organic EL device of the present invention include the following:

(1) an anode/light emitting layer/cathode;

(2) an anode/hole injecting layer/light emitting layer/cathode;

(3) an anode/light emitting layer/electron injecting layer/cathode;

(4) an anode/hole injecting layer/light emitting layer/electron injecting layer/cathode;

(5) an anode/organic semiconductor layer/light emitting layer/cathode;

(6) an anode/organic semiconductor layer/electron barrier layer/light emitting layer/cathode;

(7) an anode/organic semiconductor layer/light emitting layer/adhesion improving layer/cathode;

(8) an anode/hole injecting layer/hole transporting layer/light emitting layer/electron injecting layer/cathode;

(9) an anode/insulating layer/light emitting layer/insulating layer/cathode;

(10) an anode/inorganic semiconductor layer/insulating layer/light emitting layer/insulating layer/cathode;

(11) an anode/organic semiconductor layer/insulating layer/light emitting layer/insulating layer/cathode;

(12) an anode/insulating layer/hole injecting layer/hole transporting layer/light emitting layer/insulating layer/cathode; and

(13) an anode/insulating layer/hole injecting layer/hole transporting layer/light emitting layer/electron injecting layer/cathode.

Of those, the structure (8) is preferably used in ordinary cases. However, the structure is not limited to the foregoing.

The aromatic amine derivative of the present invention may be used in any one of the organic thin film layers of the organic EL device. The derivative can be used in a light emitting zone or a hole transporting zone. The derivative is used preferably in the hole transporting zone, or particularly preferably in a hole injecting layer, thereby making a molecule hardly crystallize and improving yields upon production of the organic EL device.

The amount of the aromatic amine derivative of the present invention to be incorporated into the organic thin film layers is preferably 30 to 100 mol %.

(2) Transparent Substrate

The organic EL device of the present invention is prepared on a transparent substrate. Here, the transparent substrate is the substrate which supports the organic EL device. It is preferable that the transparent substrate have a transmittance of light of 50% or higher in the visible region of 400 to 700 nm and be flat and smooth.

Examples of the transparent substrate include glass plates and polymer plates. Specific examples of the glass plate include plates made of soda-lime glass, glass containing barium and strontium, lead glass, aluminosilicate glass, borosilicate glass, barium borosilicate glass, and quartz. Specific examples of the polymer plate include plates made of polycarbonate resins, acrylic resins, polyethylene terephthalate, polyether sulfide, and polysulfone.

(3) Anode

The anode in the organic EL device of the present invention has the function of injecting holes into the hole transporting layer or the light emitting layer. It is effective that the anode has a work function of 4.5 eV or higher. Specific examples of the material for the anode used in the present invention include indium tin oxide (ITO) alloys, tin oxide (NESA), Indium zinc oxide (IZO), gold, silver, platinum, and copper.

The anode can be prepared by forming a thin film of the electrode material described above in accordance with a process such as the vapor deposition process and the sputtering process.

When the light emitted from the light emitting layer is obtained through the anode, it is preferable that the anode have a transmittance of the emitted light higher than 10%. It is also preferable that the sheet resistivity of the anode be several hundred Ω/□ or smaller. The thickness of the anode is, in general, selected in the range of 10 nm to 1 μm and preferably in the range of 10 to 200 nm although the preferable range may be different depending on the used material.

(4) Light Emitting Layer

The light emitting layer in the organic EL device has a combination of the following functions (1) to (3).

(1) The injecting function: the function of injecting holes from the anode or the hole injecting layer and injecting electrons from the cathode or the electron injecting layer when an electric field is applied.

(2) The transporting function: the function of transporting injected charges (i.e., electrons and holes) by the force of the electric field.

(3) The light emitting function: the function of providing the field for recombination of electrons and holes and leading to the emission of light.

However, the easiness of injection may be different between holes and electrons and the ability of transportation expressed by the mobility may be different between holes and electrons. It is preferable that either one of the charges be transferred.

When the compound of the present invention is used in a light emitting zone, a light emitting layer may be formed of the compound of the present invention alone, or the compound may be mixed with any other material before use.

A material to be mixed with the compound of the present invention to form the light emitting layer is not particularly limited as long as the material has the above preferable nature, and an arbitrary material to be used can be selected from known materials each used in the light emitting layer of an EL device.

At that time, the compound of the present invention is preferably used as a main component; a specific constitution is such that the compound of the present invention is used to account for preferably 30 to 100 mol %, or more preferably 50 to 99 mol % of the light emitting layer.

A light emitting material to be used in combination with the compound of the present invention is mainly an organic compound, and specific examples of the organic compound include the following compounds depending on a desired color tone.

First, when one desires to achieve the emission of purple light from an ultraviolet region, the examples of the organic compound include compounds each represented by the following general formula.

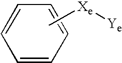

In the general formula, Xₑ represents the following compound.

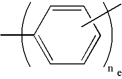

Here, nₑ represents 2, 3, 4, or 5. In addition, Yₑ represents each of the following compounds.

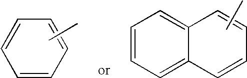

A phenyl group, phenylene group, or naphthyl group of the above compound may be substituted by one or more substituents such as an alkyl or alkoxy group having 1 to 4 carbon atoms, a hydroxy group, a sulfonyl group, a carbonyl group, an amino group, a dimethylamino group, and a diphenylamino group. In addition, those substituents may be bonded to each other to form a saturated, five- or six-membered ring. In addition, a compound in which a substituent is bonded to p-position of the phenyl, phenylene, or naphthyl group is preferable for the formation of a smooth deposited film because the substituent is favorably bonded to the position. Specific examples of the compound include the following compounds. In particular, a p-quarterphenyl derivative and a p-quinquephenyl derivative are preferable.

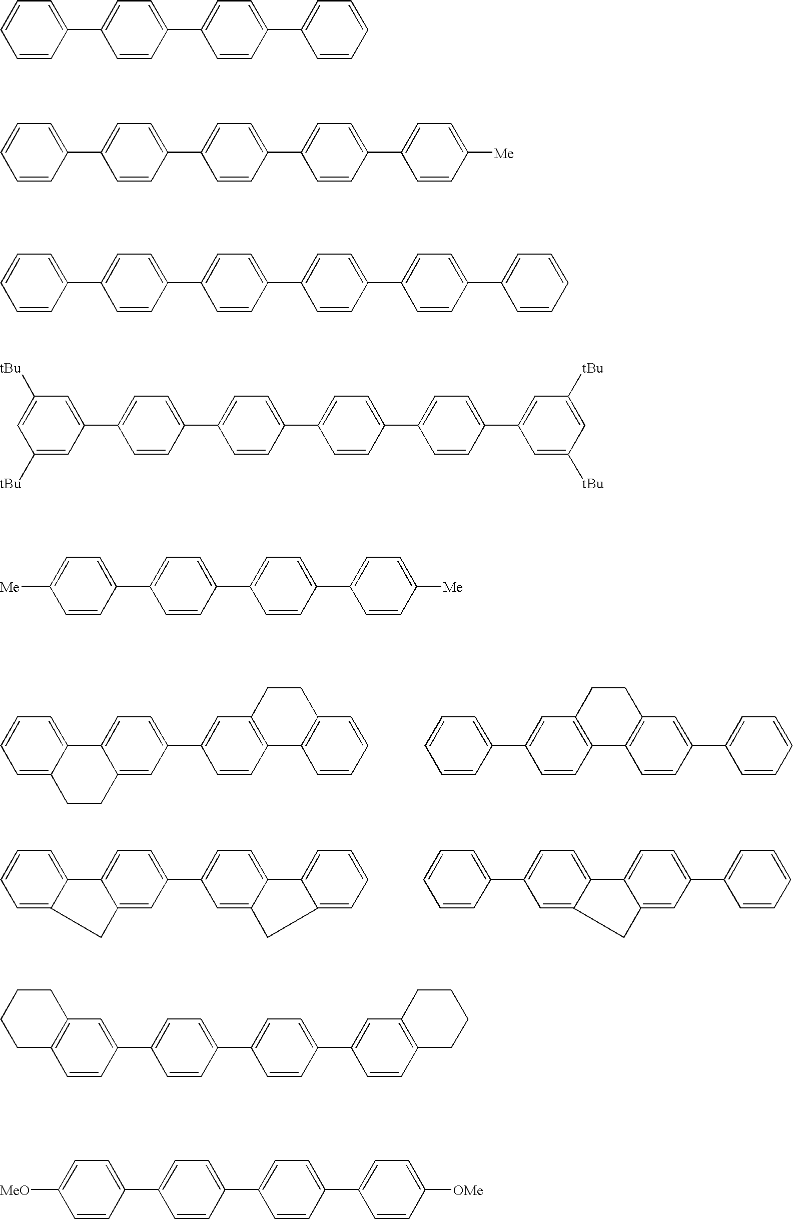

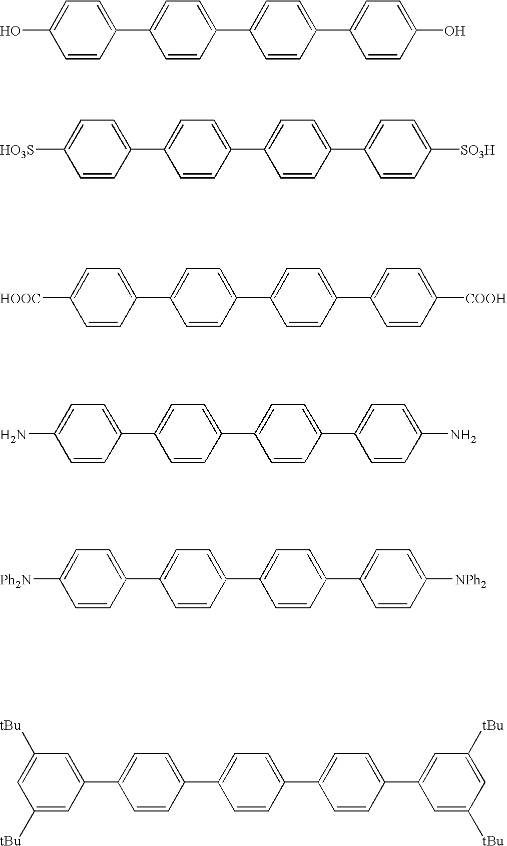

Next, when one desires to achieve the emission of blue to green light, the examples of the organic compound include a benzothiazole-based, benzimidazole-based, or benzoxazole-based fluorescent whitening agent, a metal chelated oxynoid compound, and a styrylbenzene-based compound.

Specific compound names for those compounds are disclosed in, for example, Japanese Patent Application Laid-Open No. Sho 59-194393 Useful compounds other than those described above are listed in Chemistry of Synthetic Dyes 1971, p. 628 to 637 and 640.

A compound disclosed in, for example, Japanese Patent Application Laid-Open No. Sho 63-295695 can be used as the chelated oxynoid compound. Representative examples of the compound include an 8-hydroxyquinoline-based metal complex such as tris(8-quinolinol)aluminum (hereinafter abbreviated as “Alq”) and dilithium epintridione.

In addition, a compound disclosed in, for example, European Patent No. 0319881 B or European Patent No. 0373582 B can be used as the styrylbenzene-based compound.

A distyrylpyrazine derivative disclosed in Japanese Patent Application Laid-Open No. Hei 2-252793 can also be used as a material for the light emitting layer.

In addition to the foregoing, a polyphenyl-based compound disclosed in, for example, European Patent No. 0387715 B can also be used as a material for the light emitting layer.

Further, in addition to the fluorescent whitening agent, the metal chelated oxynoid compound, the styrylbenzene-based compound, and the like described above, 12-phthaloperynone (J. Appl. Phys., vol. 27, L713 (1988)), 1,4-diphenyl-1,3-butadiene and 1,1,4,4-tetraphenyl-1,3-butadiene (each disclosed in Appl. Phys. Lett., vol. 56, L799 (1990)), a naphthalimide derivative (Japanese Patent Application Laid-Open No. Hei 2-305886), a perylene derivative (Japanese Patent Application Laid-Open No. Hei 2-189890), an oxadiazole derivative (Japanese Patent Application Laid-Open No. Hei 2-216791, or an oxadiazole derivative disclosed by Hamada et al. in the 38th Spring Meeting of The Japan Society of Applied Physics and Related Societies), an aldazine derivative (Japanese Patent Application Laid-Open No. Hei 2-220393), a pyraziline derivative (Japanese Patent Application Laid-Open No. Hei 2-220394), a cyclopentadiene derivative (Japanese Patent Application Laid-Open No. Hei 2-289675), a pyrrolopyrrole derivative (Japanese Patent Application Laid-Open No. Hei 2-296891), a styrylamine derivative (Appl. Phys. Lett., vol. 56, L799 (1990)), a coumarin-based compound (Japanese Patent Application Laid-Open No. Hei 2-191694), polymer compounds described in International Patent WO 90/13148 and Appl. Phys. Lett., vol. 58, 18, P1982 (1991), and the like can each be used as a material for the light emitting layer.

In the present invention, an aromatic dimethylidine-based compound (one disclosed in European Patent No. 0388768 B or Japanese Patent Application Laid-Open No. Hei 3-231970) is particularly preferably used as a material for the light emitting layer. Specific examples of the compound include 4,4′-bis(2,2-di-t-butylphenylvinyl)biphenyl (hereinafter abbreviated as “DTBPBBi”) and 4,4′-bis(2,2-diphenylvinyl)biphenyl (hereinafter abbreviated as “DPVBi”), and derivatives of the compounds.

The examples of the compound further include compounds each represented by a general formula (Rs-Q)₂-Al—O-L described in, for example, Japanese Patent Application Laid-Open No. Hei 5-258862 (in the above formula, L represents a hydrocarbon group having 6 to 24 carbon atoms and containing a phenyl portion, O-L represents a phenolate ligand, Q represents a substituted 8-quinolinolato ligand, and Rs represents an 8-quinolinolato ring substituent selected to prevent sterically more than two substituted 8-quinolinolato ligands from being bonded to an aluminum atom). Specific examples of the compound include bis(2-methyl-8-quinolinolato)(p-phenylphenolate)aluminum(III) (hereinafter “PC-7”) and bis(2-methyl-8-quinolinolato)(1-naphtholate)aluminum(III) (hereinafter “PC-17”).

Alternatively, the highly efficient emission of the mixture of blue light and green light can be achieved by employing doping described in, for example, Japanese Patent Application Laid-Open No. Hei 6-9953. In this case, a host is, for example, any one of the above-mentioned light emitting materials, and a dopant is, for example, a fluorescent dye capable of emitting strong light having a color ranging from a blue color to a green color, such as a coumarin-based fluorescent dye or a fluorescent dye similar to that used as the above-mentioned host. The host is specifically, for example, a light emitting material having a distyrylarylene skeleton, particularly preferably DPVBi, and the dopant is specifically, for example, a diphenylaminovinylarylene, particularly preferably, for example, N,N-diphenylaminovinylbenzene (DPAVB).

Although a light emitting layer capable of emitting white light is not particularly limited, a constitution for the layer is, for example, as follows:

(1) a constitution in which the energy level of each layer of an organic EL laminate structure is specified, and the structure is caused to emit light by utilizing tunnel injection (European Patent No. 0390551 B),

(2) a white light emitting device described as an example of a device that utilizes tunnel injection as in the case of the above item (1) (Japanese Patent Application Laid-Open No. Hei 3-230584),

(3) a light emitting layer having a two-layered structure (Japanese Patent Application Laid-Open No. Hei 2-220396 or Japanese Patent Application Laid-Open No. Hei 2-216790),

(4) a constitution in which a light emitting layer is divided into multiple portions, and the respective portions are constituted of materials different from each other in luminous wavelength (Japanese Patent Application Laid-Open No. Hei 4-51491),

(5) a constitution in which a blue light emitting body (showing a fluorescent peak at 380 to 480 nm) and a green light emitting body (showing a fluorescent peak at 480 to 580 nm) are laminated, and, furthermore, a red fluorescent material is incorporated into the laminate (Japanese Patent Application Laid-Open No. Hei 6-207170), or

(6) a constitution in which a blue light emitting layer contains a blue fluorescent dye, and a green light emitting layer has a region containing a red fluorescent dye and further contains a green fluorescent material (Japanese Patent Application Laid-Open No. Hei 7-142169).

Of those, the constitution described in the above item (5) is preferably used.

Examples of the red fluorescent material are shown below.

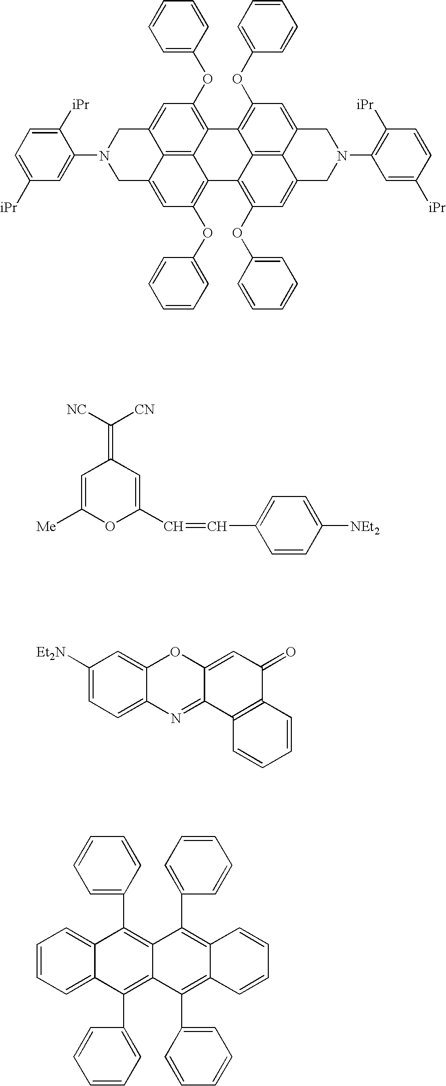

A known method such as a vapor deposition method, a spin coating method, or an LB method is applicable to the formation of the light emitting layer using any one of the above-mentioned materials. The light emitting layer is particularly preferably a molecular deposit film. The term “molecular deposit film” as used herein refers to a thin film formed by the deposition of a material compound in a vapor phase state, or a film formed by the solidification of a material compound in a solution state or a liquid phase state. The molecular deposit film can be typically distinguished from a thin film formed by the LB method (molecular accumulation film) on the basis of differences between the films in aggregation structure and higher order structure, and functional differences between the films caused by the foregoing differences.

In addition, as disclosed in Japanese Patent Application Laid-Open No. Sho 57-51781, the light emitting layer can also be formed by: dissolving a binder such as a resin and a material compound in a solvent to prepare a solution; and forming a thin film from the prepared solution by the spin coating method or the like.

The thickness of the light emitting layer thus formed is not particularly limited, and can be appropriately selected depending on circumstances; the thickness is preferably in the range of 5 nm to 5 μm in ordinary cases. The light emitting layer may be constituted of a single layer composed of one or two or more kinds of the above-mentioned materials, or a light emitting layer composed of a compound different from the compound of which the foregoing light emitting layer is composed may be laminated on the foregoing light emitting layer.

When the compound of the present invention is used in a light emitting zone, the light emitting layer may be constituted of a single layer composed of one or two or more kinds of the above-mentioned materials as long as the layer contains the compound of the present invention.

In addition, a phosphorescent compound can also be used as a light emitting material. A compound containing a carbazole ring as a host material is preferable as the phosphorescent compound. The dopant is a compound capable of emitting light from a triplet exciton, and is not particularly limited as long as light is emitted from a triplet exciton, a metal complex containing at least one metal selected from the group consisting of Ir, Ru, Pd, Pt, Os, and Re is preferable, and a porphyrin metal complex or an orthometalated metal complex is preferable.

A host composed of a compound containing a carbazole ring and suitable for phosphorescence is a compound having a function of causing a phosphorescent compound to emit light as a result of the occurrence of energy transfer from the excited state of the host to the phosphorescent compound. A host compound is not particularly limited as long as it is a compound capable of transferring exciton energy to a phosphorescent compound, and can be appropriately selected in accordance with a purpose. The host compound may have, for example, an arbitrary heterocyclic ring in addition to a carbazole ring.

Specific examples of such a host compound include a carbazole derivative, a triazole derivative, an oxazole derivative, an oxadiazole derivative, an imidazole derivative, a polyarylalkane derivative, a pyrazoline derivative, a pyrazolone derivative, a phenylene diamine derivative, an aryl amine derivative, an amino substituted chalcone derivative, a styrylanthracene derivative, a fluorenone derivative, a hydrazone derivative, a stilbene derivative, a silazane derivative, an aromatic tertiary amine compound, a styryl amine compound, an aromatic dimethylidene-based compound, a porphyrin-based compound, an anthraquinodimethane derivative, an anthrone derivative, a diphenylquinone derivative, a thiopyranedioxide derivative, a carbodiimide derivative, a fluorenilidene methane derivative, a distyryl pyrazine derivative, a heterocyclic tetracarboxylic anhydride such as naphthaleneperylene, a phthalocyanine derivative, various metal complex typified by a metal complex of an 8-quinolinol derivative or a metal complex having metal phthalocyanine, benzooxazole, or benzothiazole as a ligand, polysilane-based compounds, a poly(N-vinylcarbazole) derivative, an aniline-based copolymer, a conductive high molecular weight oligomer such as a thiophene oligomer or polythiophene, polymer compounds such as a polythiophene derivative, a polyphenylene derivative, a polyphenylene vinylene derivative, and a polyfluorene derivative. One of the host materials may be used alone, or two or more of them may be used in combination.

Specific examples thereof include the compounds as described below.

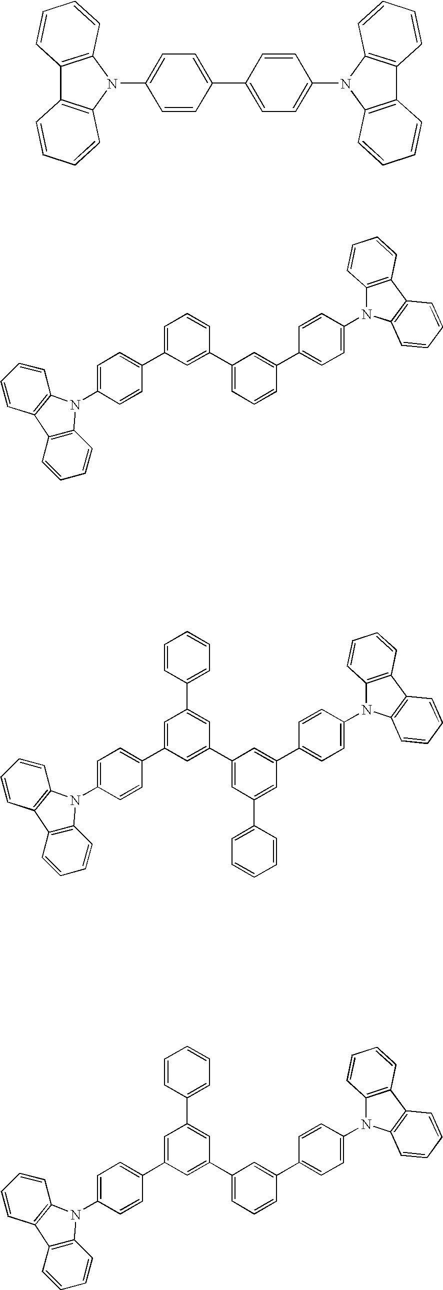

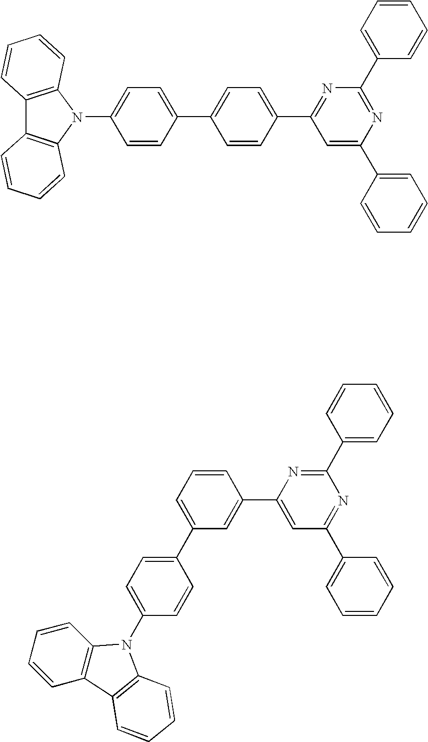

A phosphorescent dopant is a compound capable of emitting light from a triplet exciton. The dopant, which is not particularly limited as long as light is emitted from a triplet exciton, is preferably a metal complex containing at least one metal selected from the group consisting of Ir, Ru, Pd, Pt, Os, and Re, and is preferably a porphyrin metal complex or an orthometalated metal complex. A porphyrin platinum complex is preferable as the porphyrin metal complex. One kind of a phosphorescent compound may be used alone, or two or more kinds of phosphorescent compounds may be used in combination.

Any one of various ligands can be used for forming an orthometalated metal complex. Examples of a preferable ligand include a 2-phenylpyridine derivative, a 7,8-benzoquinoline derivative, a 2-(2-thienyl)pyridine derivative, a 2-(1-naphthyl)pyridine derivative, and a 2-phenylquinoline derivative. Each of those derivatives may have a substituent as required. A fluoride of any one of those derivatives, or one obtained by introducing a trifluoromethyl group into any one of those derivatives is a particularly preferable blue-based dopant. The metal complex may further include a ligand other than the above-mentioned ligands such as acetylacetonate or picric acid as an auxiliary ligand.

The content of the phosphorescent dopant in the light emitting layer is not particularly limited, and can be appropriately selected in accordance with a purpose. The content is, for example, 0.1 to 70 mass %, and is preferably 1 to 30 mass %. When the content of the phosphorescent compound is less than 0.1 mass %, the intensity of emitted light is weak, and an effect of the incorporation of the compound is not sufficiently exerted. When the content exceeds 70 mass %, a phenomenon referred to as concentration quenching becomes remarkable, and device performance reduces.

In addition, the light emitting layer may contain a hole transporting material, an electron transporting material, or a polymer binder as required.

Further, the thickness of the light emitting layer is preferably 5 to 50 nm, more preferably 7 to 50 nm, or most preferably 10 to 50 nm. When the thickness is less than 5 nm, it becomes difficult to form the light emitting layer, so the adjustment of chromaticity may be difficult. When the thickness exceeds 50 nm, the driving voltage may increase.

(5) Hole Injecting and Transporting Layer (Hole Transporting Zone)

The hole injecting and transporting layer is a layer which helps injection of holes into the light emitting layer and transports the holes to the light emitting region. The layer exhibits a great mobility of holes and, in general, has an ionization energy as small as 5.6 eV or smaller. For such the hole injecting and transporting layer, a material which transports holes to the light emitting layer under an electric field of a smaller strength is preferable. A material which exhibits, for example, a mobility of holes of at least 10⁻⁴ m²/V sec under application of an electric field of 10⁴ to 10⁶ V/cm is preferable.

When the aromatic amine derivative of the present invention is used in the hole transporting zone, the aromatic amine derivative of the present invention may be used alone or as a mixture with other materials for forming the hole injecting and transporting layer.

The material which can be used for forming the hole injecting and transporting layer as a mixture with the aromatic amine derivative of the present invention is not particularly limited as long as the material has a preferable property described above. The material can be arbitrarily selected from materials which are conventionally used as the charge transporting material of holes in photoconductive materials and known materials which are used for the hole injecting and transporting layer in organic EL devices. In the present invention, a material which has hole transporting ability and can be used in the transporting zone is referred to as a hole transporting material.

Specific examples include: a triazole derivative (see, for example, U.S. Pat. No. 3,112,197); an oxadiazole derivative (see, for example, U.S. Pat. No. 3,189,447); an imidazole derivative (see, for example, JP-B-37-16096); a polyarylalkane derivative (see, for example, U.S. Pat. No. 3,615,402, U.S. Pat. No. 3,820,989, U.S. Pat. No. 3,542,544, JP-B-45-555, JP-B-51-10983, JP-A-51-93224, JP-A-55-17105, JP-A-56-4148, JP-A-55-108667, JP-A-55-156953, and JP-A-56-36656); a pyrazoline derivative and a pyrazolone derivative (see, for example, U.S. Pat. No. 3,180,729, U.S. Pat. No. 4,278,746, JP-A-5S-88064, JP-A-55-88065, JP-A-49-105537, JP-A-55-51086, JP-A-56-80051, JP-A-56-88141, JP-A-57-45545, JP-A-54-112637, and JP-A-55-74546); a phenylenediamine derivative (see, for example, U.S. Pat. No. 3,615,404, JP-B-51-10105, JP-B-46-3712, JP-B-47-25336, and JP-A-54-119925); an arylamine derivative (see, for example, U.S. Pat. No. 3,567,450, U.S. Pat. No. 3,240,597, U.S. Pat. No. 3,658,520, U.S. Pat. No. 4,232,103, U.S. Pat. No. 4,175,961, U.S. Pat. No. 4,012,376, JP-B-49-35702, JP-B-39-27577, JP-A-55-144250, JP-A-56-119132, JP-A-56-22437, and DE 1,110,518); an amino-substituted chalcone derivative (see, for example, U.S. Pat. No. 3,526,501); an oxazole derivative (those disclosed in U.S. Pat. No. 3,257,203); a styrylanthracene derivative (see, for example, JP-A-56-46234); a fluorenone derivative (see, for example, JP-A-54-110837); a hydrazone derivative (see, for example, U.S. Pat. No. 3,717,462, JP-A-54-59143, JP-A-55-52063, JP-A-55-52064, JP-A-55-46760, JP-A-57-11350, JP-A-57-148749, and JP-A-2-311591); a stilbene derivative (see, for example, JP-A-61-210363, JP-A-61-228451, JP-A-61-14642, JP-A-61-72255, JP-A-62-47646, JP-A-62-36674, JP-A-62-10652, JP-A-62-30255, JP-A-60-93445, JP-A-60-94462, JP-A-60-174749, and JP-A-60-175052); and a silazane derivative (U.S. Pat. No. 4,950,950); a polysilane-based copolymer (JP-A-2-204996); an aniline-based copolymer (JP-A-2-282263).

In addition to the above-mentioned materials which can be used as the material for the hole injecting and transporting layer, a porphyrin compound (those disclosed in, for example, JP-A-63-295695); an aromatic tertiary amine compound and a styrylamine compound (see, for example, U.S. Pat. No. 4,127,412, JP-A-53-27033, JP-A-54-58445, JP-A-55-79450, JP-A-55-144250, JP-A-56-119132, JP-A-61-295558, JP-A-61-98353, and JP-A-63-295695) are preferable, and aromatic tertiary amines are particularly preferable.

Further examples of aromatic tertiary amine compounds include compounds having two fused aromatic rings in the molecule such as 4,4′-bis(N-(1-naphthyl)-N-phenylamino)-biphenyl (hereinafter referred to as NPD) as disclosed in U.S. Pat. No. 5,061,569, and a compound in which three triphenylamine units are bonded together in a star-burst shape, such as 4,4′,4″-tris(N-(3-methylphenyl)-N-phenylamino)triphenylamine (hereinafter referred to as MTDATA) as disclosed in JP-A-4-308688.

Further, in addition to the aromatic dimethylidine-based compounds described above as the material for the light emitting layer, inorganic compounds such as Si of the p-type and SiC of the p-type can also be used as the material for the hole injecting and transporting layer.

The hole injecting and transporting layer can be formed by forming a thin layer from the aromatic amine derivative of the present invention in accordance with a known process such as the vacuum vapor deposition process, the spin coating process, the casting process, and the LB process. The thickness of the hole injecting and transporting layer is not particularly limited. In general, the thickness is 5 nm to 5 μm. The hole injecting and transporting layer may be constituted of a single layer containing one or more materials described above or may be a laminate constituted of hole injecting and transporting layers containing materials different from the materials of the hole injecting and transporting layer described above as long as the aromatic amine derivative of the present invention is incorporated in the hole injecting and transporting zone.

Further, an organic semiconductor layer may be disposed as a layer for helping the injection of holes or electrons into the light emitting layer. As the organic semiconductor layer, a layer having a conductivity of 10⁻¹⁰ S/cm or higher is preferable. As the material for the organic semiconductor layer, oligomers containing thiophene, and conductive oligomers such as oligomers containing arylamine and conductive dendrimers such as dendrimers containing arylamine which are disclosed in JP-A-08-193191, can be used.

(6) Electron Injecting and Transporting Layer

Next, the electron injecting and transporting layer is a layer which helps injection of electrons into the light emitting layer, transports the electrons to the light emitting region, and exhibits a great mobility of electrons. The adhesion improving layer is an electron injecting layer including a material exhibiting particularly improved adhesion with the cathode.

In addition, it is known that, in an organic EL device, emitted light is reflected by an electrode (cathode in this case), so emitted light directly extracted from an anode and emitted light extracted via the reflection by the electrode interfere with each other. The thickness of an electron transporting layer is appropriately selected from the range of several nanometers to several micrometers in order that the interference effect may be effectively utilized. When the thickness is particularly large, an electron mobility is preferably at least 10⁻⁵ m²/Vs or more upon application of an electric field of 10⁴ to 10⁶ V/cm in order to avoid an increase in voltage.

A metal complex of 8-hydroxyquinoline or of a derivative of 8-hydroxyquinoline, or an oxadiazole derivative is suitable as a material to be used in an electron injecting layer. Specific examples of the metal complex of 8-hydroxyquinoline or of a derivative of 8-hydroxyquinoline that can be used as an electron injecting material include metal chelate oxynoid compounds each containing a chelate of oxine (generally 8-quinolinol or 8-hydroxyquinoline), such as tris(8-quinolinol)aluminum.

On the other hand, examples of the oxadiazole derivative include electron transfer compounds represented by the following general formulae:

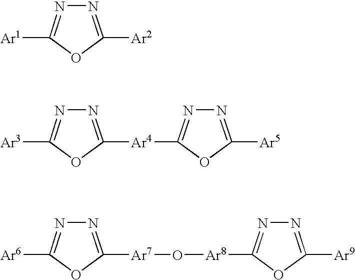

(where: Ar¹, Ar², Ar³, Ar⁵, Ar⁶ and Ar⁹ each represent a substituted or unsubstituted aryl group and may represent the same group or different groups. Ar⁴, Ar⁷ and Ar⁸ each represent a substituted or unsubstituted arylene group and may represent the same group or different groups.)

Examples of the aryl group include a phenyl group, a biphenylyl group, an anthryl group, a perylenyl group, and a pyrenyl group. Examples of the arylene group include a phenylene group, a naphthylene group, a biphenylene group, an anthrylene group, a perylenylene group, and a pyrenylene group. Examples of the substituent include alkyl groups each having 1 to 10 carbon atoms, alkoxyl groups each having 1 to 10 carbon atoms, and a cyano group. As the electron transfer compound, compounds which can form thin films are preferable.

Examples of the electron transfer compounds described above include the following.

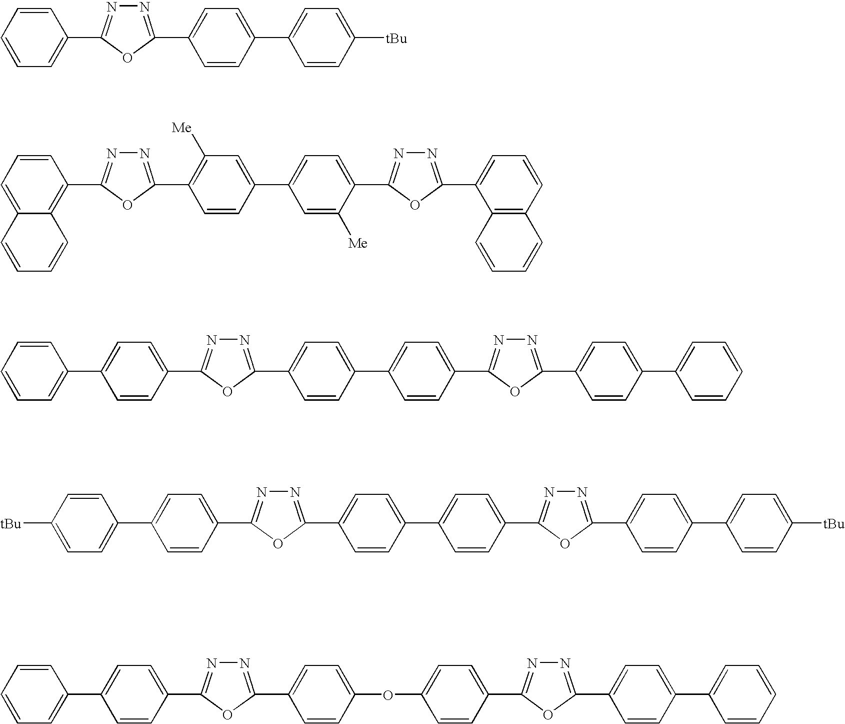

Further, materials represented by the following general formulae (A) to (F) can be used in an electron injecting layer and an electron transporting layer.

Nitrogen-containing heterocyclic ring derivative represented by the general formula (A) or (B).

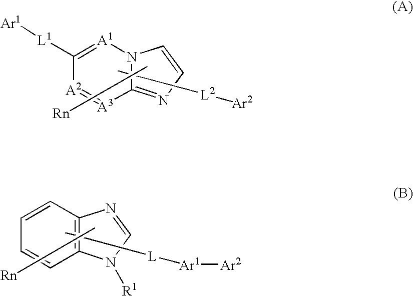

(In the general formulae (A) and (B), A¹ to A³ each independently represent a nitrogen atom or a carbon atom.

Ar¹ represents a substituted or unsubstituted aryl group having 6 to 60 ring carbon atoms, or a substituted or unsubstituted heteroaryl group having 3 to 60 ring carbon atoms, Ar² represents a hydrogen atom, a substituted or unsubstituted aryl group having 6 to 60 ring carbon atoms, a substituted or unsubstituted heteroaryl group having 3 to 60 ring carbon atoms, a substituted or unsubstituted alkyl group having 1 to 20 carbon atoms, or a substituted or unsubstituted alkoxy group having 1 to 20 carbon atoms, or a divalent group of any one of them provided that one of Ar₁ and Ar² represents a substituted or unsubstituted fused ring group having 10 to 60 ring carbon atoms, a substituted or unsubstituted monohetero fused ring group having 3 to 60 ring carbon atoms, or a divalent group of any one of them.

L¹, L², and L each independently represent a single bond, a substituted or unsubstituted arylene group having 6 to 60 ring carbon atoms, a substituted or unsubstituted heteroarylene group having 3 to 60 ring carbon atoms, or a substituted or unsubstituted fluorenylene group.

R represents a hydrogen atom, a substituted or unsubstituted aryl group having 6 to 60 ring carbon atoms, a substituted or unsubstituted heteroaryl group having 3 to 60 ring carbon atoms, a substituted or unsubstituted alkyl group having 1 to 20 carbon atoms, or a substituted or unsubstituted alkoxy group having 1 to 20 carbon atoms. n represents an integer of 0 to 5, and, when n represents 2 or more, multiple R's may be identical to or different from each other, and multiple R groups adjacent to each other may be bonded to each other to form a carbocyclic aliphatic ring or a carbocyclic aromatic ring.

R¹ represents a hydrogen atom, a substituted or unsubstituted aryl group having 6 to 60 ring carbon atoms, a substituted or unsubstituted heteroaryl group having 3 to 60 ring carbon atoms, a substituted or unsubstituted alkyl group having 1 to 20 carbon atoms, a substituted or unsubstituted alkoxy group having 1 to 20 carbon atoms, or a group represented by -L-Ar¹—Ar².)

Nitrogen-containing heterocyclic ring derivative represented by the general formula (C).
HAr-L-Ar¹—Ar² (C)

where, HAr represents a nitrogen-containing heterocyclic ring which has 3 to 40 carbon atoms and may have a substituent; L represents a single bond, an arylene group which has 6 to 60 carbon atoms and may have a substituent, a heteroarylene group which has 3 to 60 carbon atoms and may have a substituent, or a fluorenylene group which may have a substituent; Ar¹ represents a divalent aromatic hydrocarbon group which has 6 to 60 carbon atoms and may have a substituent; and Ar² represents an aryl group which has 6 to 60 carbon atoms and may have a substituent, or a heteroaryl group which has 3 to 60 carbon atoms and may have a substituent.

Silacyclopentadiene derivative represented by the general formula (D)

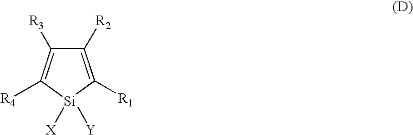

where; X and Y each independently represent a saturated or unsaturated hydrocarbon group having 1 to 6 carbon atoms, an alkoxy group, an alkenyloxy group, an alkynyloxy group, a hydroxy group, a substituted or unsubstituted aryl group, or a substituted or unsubstituted heterocycle, or X and Y are bonded to each other to form a structure as a saturated or unsaturated ring; and R₁ to R₄ each independently represent hydrogen, a halogen atom, a substituted or unsubstituted alkyl group having 1 to 6 carbon atoms, an alkoxy group, an aryloxy group, a perfluoroalkyl group, a perfluoroalkoxy group, an amino group, an alkylcarbonyl group, an arylcarbonyl group, an alkoxycarbonyl group, an aryloxycarbonyl group, an azo group, an alkylcarbonyloxy group, an arylcarbonyloxy group, an alkoxycarbonyloxy group, an aryloxycarbonyloxy group, a sulfinyl group, a sulfonyl group, a sulfanyl group, a silyl group, carbamoyl group, an aryl group, a heterocyclic group, an alkenyl group, an alkynyl group, a nitro group, a formyl group, a nitroso group, a formyloxy group, an isocyano group, a cyanate group, an isocyanate group, a thiocyanate group, an isothiocyanate group, or a cyano group, or, when two or more of R₁ to R₄ are adjacent to each other, they may form a structure in which a substituted or unsubstituted ring is fused.

Borane derivative represented by the general formula (E),

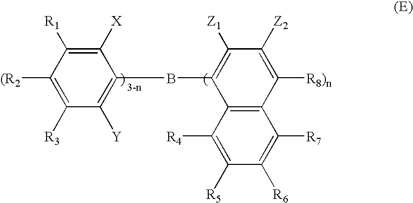

where: R₁ to R₈ and Z₂ each independently represent a hydrogen atom, a saturated or unsaturated hydrocarbon group, an aromatic group, a heterocyclic group, a substituted amino group, a substituted boryl group, an alkoxy group, or an aryloxy group; X, Y, and Z₁ each independently represent a saturated or unsaturated hydrocarbon group, an aromatic group, a heterocyclic group, a substituted amino group, an alkoxy group, or an aryloxy group; substituents of Z₁ and Z₂ may be bonded to each other to form a fused ring; and n represents an integer of 1 to 3, and, when n represents 2 or more, Z₁'s may be different from each other provided that the case where n represents 1, X, Y, and R₂ each represent a methyl group, R₈ represents a hydrogen atom or a substituted boryl group and the case where n represents 3 and Z₁'s each represent a methyl group are excluded.

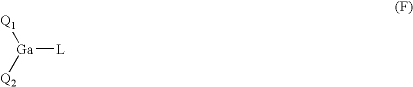

where: Q¹ and Q² each independently represent a ligand represented by the following general formula (G); and L represents a halogen atom, a substituted or unsubstituted alkyl group, a substituted or unsubstituted cycloalkyl group, a substituted or unsubstituted aryl group, a substituted or unsubstituted heterocyclic ring group, —OR¹ where R¹ represents a hydrogen atom, a substituted or unsubstituted alkyl group, a substituted or unsubstituted cycloalkyl group, a substituted or unsubstituted aryl group, or a substituted or unsubstituted heterocyclic ring group, or a ligand represented by —O—Ga-Q³ (Q⁴) where Q³ and Q⁴ are identical to Q¹ and Q², respectively.

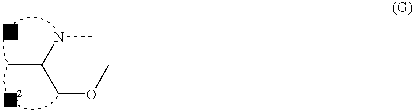

(where rings A¹ and A² are six-membered aryl ring structures which are fused with each other and each of which may have a substituent.)

The metal complex behaves strongly as an n-type semiconductor, and has a large electron injecting ability. Further, generation energy upon formation of the complex is low. As a result, the metal and the ligand of the formed metal complex are bonded to each other so strongly that the fluorescent quantum efficiency of the complex as a light emitting material improves.

Specific examples of a substituent in the rings A¹ and A² which each form a ligand in the general formula (G) include: a halogen atom such as chlorine, bromine, iodine, or fluorine; a substituted or unsubstituted alkyl group such as a methyl group, an ethyl group, a propyl group, a butyl group, an s-butyl group, a t-butyl group, a pentyl group, a hexyl group, a heptyl group, an octyl group, a stearyl group, or trichloromethyl group; a substituted or unsubstituted aryl group such as a phenyl group, a naphthyl group, a 3-methylphenyl group, a 3-methoxyphenyl group, a 3-fluorophenyl group, a 3-trichloromethylphenyl group, a 3-trifluoromethylphenyl group, or a 3-nitrophenyl group; a substituted or unsubstituted alkoxy group such as a methoxy group, an n-butoxy group, a t-butoxy group, a trichloromethoxy group, a trifluoroethoxy group, a pentafluoropropoxy group, a 2,2,3,3-tetrafluoropropoxy group, an 1,1,1,3,3,3-hexafluoro-2-propoxy group, or a 6-(perfluoroethyl)hexyloxy group; a substituted or unsubstituted aryloxy group such as a phenoxy group, a p-nitrophenoxy group, p-t-butylphenoxy group, a 3-fluorophenoxy group, a pentafluorophenyl group, or a 3-trifluoromethylphenoxy group; a substituted or unsubstituted alkylthio group such as a methylthio group, an ethylthio group, a t-butylthio group, a hexylthio group, an octylthio group, or a trifluoromethylthio group; a substituted or unsubstituted arylthio group such as a phenylthio group, a p-nitrophenylthio group, a p-t-butylphenylthio group, a 3-fluorophenylthio group, a pentafluorophenylthio group, or a 3-trifluoromethylphenylthio group; a cyano group; a nitro group; an amino group; a mono-substituted or di-substituted amino group such as a methylamino group, a diethylamino group, an ethylamino group, a diethylamino group, a dipropylamino group, a dibutylamino group, or a diphenylamino group; an acylamino group such as a bis(acetoxymethyl)amino group, a bis(acetoxyethyl)amino group, a bis(acetoxypropyl)amino group, or a bis(acetoxybutyl)amino group; a hydroxy group; a siloxy group; an acyl group; a carbamoyl group such as a methylcarbamoyl group, a dimethylcarbamoyl group, an ethylcarbamoyl group, a diethylcarbamoyl group, a propylcarbamoyl group, a butylcarbamoyl group, or a phenylcarbamoyl group; a carboxylic acid group; a sulfonic acid group; an imide group; a cycloalkyl group such as a cyclopentane group, or a cyclohexyl group; an aryl group such as a phenyl group, a naphthyl group, a biphenylyl group, an anthryl group, a phenanthryl group, a fluorenyl group, or a pyrenyl group; and a heterocyclic group such as a pyridinyl group, a pyrazinyl group, a pyrimidinyl group, a pyridazinyl group, a triazinyl group, an indolinyl group, a quinolinyl group, an acridinyl group, a pyrrolidinyl group, a dioxanyl group, a piperidinyl group, a morpholidinyl group, a piperazinyl group, a triathinyl group, a carbazolyl group, a furanyl group, a thiophenyl group, an oxazolyl group, an oxadiazolyl group, a benzoxazolyl group, a thiazolyl group, a thiadiazolyl group, a benzothiazolyl group, a triazolyl group, an imidazolyl group, a benzoimidazolyl group, or a puranyl group. In addition, the above-mentioned substituents may be bound to each other to further form a six-membered aryl ring or a heterocycle.

A preferable embodiment of the organic EL device of the present invention includes an element including a reducing dopant in the region of electron transport or in the interfacial region of the cathode and the organic layer. The reducing dopant is defined as a substance which can reduce a compound having the electron-transporting property. Various compounds can be used as the reducing dopant as long as the compounds have a certain reductive property. For example, at least one substance selected from the group consisting of alkali metals, alkaline earth metals, rare earth metals, alkali metal oxides, alkali metal halides, alkaline earth metal oxides, alkaline earth metal halides, rare earth metal oxides, rare earth metal halides, organic complexes of alkali metals, organic complexes of alkaline earth metals, and organic complexes of rare earth metals can be preferably used.

More specifically, examples of the reducing dopant include substances having a work function of 2.9 eV or smaller, specific examples of which include at least one alkali metal selected from the group consisting of Li (the work function: 2.9 eV), Na (the work function: 2.36 eV), K (the work function: 2.28 eV), Rb (the work function: 2.16 eV), and Cs (the work function: 1.95 eV) and at least one alkaline earth metal selected from the group consisting of Ca (the work function: 2.9 eV), Sr (the work function: 2.0 to 2.5 eV), and Ba (the work function: 2.52 eV). Among the above-mentioned substances, at least one alkali metal selected from the group consisting of K, Rb, and Cs is more preferable, Rb and Cs are still more preferable, and Cs is most preferable as the reducing dopant. Those alkali metals have great reducing ability, and the luminance of the emitted light and the life time of the organic EL device can be increased by addition of a relatively small amount of the alkali metal into the electron injecting zone. As the reducing dopant having a work function of 2.9 eV or smaller, combinations of two or more alkali metals thereof are also preferable. Combinations having Cs such as the combinations of Cs and Na, Cs and K, Cs and Rb, and Cs, Na, and K are more preferable. The reducing ability can be efficiently exhibited by the combination having Cs. The luminance of emitted light and the life time of the organic EL device can be increased by adding the combination having Cs into the electron injecting zone.

The present invention may further include an electron injecting layer which is composed of an insulating material or a semiconductor and disposed between the cathode and the organic layer. At this time, leak of electric current can be effectively prevented by the electron injecting layer and the electron injecting property can be improved. As the insulating material, at least one metal compound selected from the group consisting of alkali metal chalcogenides, alkaline earth metal chalcogenides, alkali metal halides, and alkaline earth metal halides is preferable. It is preferable that the electron injecting layer be composed of the above-mentioned substance such as the alkali metal chalcogenide since the electron injecting property can be further improved. Preferable examples of the alkali metal chalcogenide include Li₂O, K₂O, Na₂S, Na₂Se, and Na₂O. To be specific, preferable examples of the alkaline earth metal chalcogenide include CaO, BaO, SrO, BeO, BaS, and CaSe. Preferable examples of the alkali metal halide include LiF, NaF, KF, LiCl, KCl, and NaCl. Preferable examples of the alkaline earth metal halide include fluorides such as CaF₂, BaF₂, SrF₂, MgF₂, and BeF₂ and halides other than the fluorides.

Examples of the semiconductor composing the electron-transporting layer include oxides, nitrides, and oxide nitrides of at least one element selected from Ba, Ca, Sr, Yb, Al, Ga, In, Li, Na, Cd, Mg, Si, Ta, Sb, and Zn used alone or in combination of two or more. It is preferable that the inorganic compound composing the electron-transporting layer form a crystallite or amorphous insulating thin film. When the electron injecting layer is composed of the insulating thin film described above, a more uniform thin film can be formed, and defects of pixels such as dark spots can be decreased. Examples of the inorganic compound include alkali metal chalcogenides, alkaline earth metal chalcogenides, alkali metal halides, and alkaline earth metal halides which are described above.

(7) Cathode

As the cathode, a material such as a metal, an alloy, a conductive compound, or a mixture of those materials which has a small work function (4 eV or smaller) is used because the cathode is used for injecting electrons to the electron injecting and transporting layer or the light emitting layer. Specific examples of the electrode material include sodium, sodium-potassium alloys, magnesium, lithium, magnesium-silver alloys, aluminum/aluminum oxide, aluminum-lithium alloys, indium, and rare earth metals.

The cathode can be prepared by forming a thin film of the electrode material described above in accordance with a process such as the vapor deposition process and the sputtering process.

When the light emitted from the light emitting layer is obtained through the cathode, it is preferable that the cathode have a transmittance of the emitted light higher than 10%.

It is also preferable that the sheet resistivity of the cathode be several hundred Ω/□ or smaller. The thickness of the cathode is, in general, selected in the range of 10 nm to 1 μm and preferably in the range of 50 to 200 nm.

(8) Insulating Layer

Defects in pixels tend to be formed in organic EL device due to leak and short circuit since an electric field is applied to ultra-thin films. To prevent the formation of the defects, a layer of a thin film having an insulating property may be inserted between the pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. Mixtures and laminates of the above-mentioned compounds may also be used.

(9) Method of Producing the Organic EL Device

To prepare the organic EL device of the present invention, the anode and the light emitting layer, and, where necessary, the hole injecting and the transporting layer and the electron injecting and transporting layer are formed in accordance with the illustrated process using the illustrated materials, and the cathode is formed in the last step. The organic EL device may also be prepared by forming the above-mentioned layers in the order reverse to that described above, i.e., the cathode being formed in the first step and the anode in the last step.

Hereinafter, an embodiment of the process for preparing an organic EL device having a construction in which an anode, a hole injecting layer, a light emitting layer, an electron injecting layer, and a cathode are disposed successively on a transparent substrate will be described.

On a suitable transparent substrate, a thin film made of a material for the anode is formed in accordance with the vapor deposition process or the sputtering process so that the thickness of the formed thin film is 1 um or smaller and preferably in the range of 10 to 200 nm. The formed thin film is used as the anode. Then, a hole injecting layer is formed on the anode. The hole injecting layer can be formed in accordance with the vacuum vapor deposition process, the spin coating process, the casting process, or the LB process, as described above. The vacuum vapor deposition process is preferable since a uniform film can be easily obtained and the possibility of formation of pin holes is small. When the hole injecting layer is formed in accordance with the vacuum vapor deposition process, in general, it is preferable that the conditions be suitably selected in the following ranges: the temperature of the source of the deposition: 50 to 450° C.; the vacuum: 10⁻⁷ to 10⁻³ Torr; the rate of deposition: 0.01 to 50 nm/second; the temperature of the substrate: −50 to 300° C. and the thickness of the film: 5 nm to 5 μm; although the conditions of the vacuum vapor deposition are different depending on the compound to be used (i.e., the material for the hole injecting layer) and the crystal structure and the recombination structure of the target hole injecting layer.

Then, the light emitting layer is formed on the hole injecting layer formed above. A thin film of the organic light emitting material can be formed by using a desired organic light emitting material in accordance with a process such as the vacuum vapor deposition process, the sputtering process, the spin coating process, or the casting process, and the formed thin film is used as the light emitting layer. The vacuum vapor deposition process is preferable since a uniform film can be easily obtained and the possibility of formation of pin holes is small. When the light emitting layer is formed in accordance with the vacuum vapor deposition process, in general, the conditions of the vacuum vapor deposition process can be selected in the same ranges as those described for the vacuum vapor deposition of the hole injecting layer, although the conditions are different depending on the used compound.

Next, an electron injecting layer is formed on the light emitting layer formed above. Similarly to the hole injecting layer and the light emitting layer, it is preferable that the electron injecting layer be formed in accordance with the vacuum vapor deposition process since a uniform film must be obtained. The conditions of the vacuum vapor deposition can be selected in the same ranges as those described for the vacuum vapor deposition of the hole injecting layer and the light emitting layer.

When the vapor deposition process is used, the aromatic amine derivative of the present invention can be deposited by vapor in combination with other materials, although the situation may be different depending on which layer in the light emitting zone or in the hole transporting zone includes the compound. When the spin coating process is used, the compound can be incorporated into the formed layer by using a mixture of the compound with other materials.

A cathode is formed on the electron injecting layer formed above in the last step, and an organic EL device can be obtained.

The cathode is made of a metal and can be formed in accordance with the vacuum vapor deposition process or the sputtering process It is preferable that the vacuum vapor deposition process be used in order to prevent formation of damages on the lower organic layers during the formation of the film.

In the above-mentioned preparation of the organic EL device, it is preferable that the above-mentioned layers from the anode to the cathode be formed successively while the preparation system is kept in a vacuum after being evacuated once.

The method of forming the layers in the organic EL device of the present invention is not particularly limited. A conventionally known process such as the vacuum vapor deposition process or the spin coating process can be used. The organic thin film layer which is used in the organic EL device of the present invention and includes the compound represented by general formula (1) described above can be formed in accordance with a known process such as the vacuum vapor deposition process or the molecular beam epitaxy process (the MBE process) or, using a solution prepared by dissolving the compounds into a solvent, in accordance with a coating process such as the dipping process, the spin coating process, the casting process, the bar coating process, or the roll coating process.

The thickness of each layer in the organic thin film layer in the organic EL device of the present invention is not particularly limited. In general, an excessively thin layer tends to have defects such as pin holes, and an excessively thick layer requires a high applied voltage to decrease the efficiency. Therefore, a thickness in the range of several nanometers to 1 μm is preferable.

The organic EL device which can be prepared as described above emits light when a direct voltage of 5 to 40 V is applied in the condition that the polarity of the anode is positive (+) and the polarity of the cathode is negative (−). When the polarity is reversed, no electric current is observed and no light is emitted at all. When an alternating voltage is applied to the organic EL device, the uniform light emission is observed only in the condition that the polarity of the anode is positive and the polarity of the cathode is negative. When an alternating voltage is applied to the organic EL device, any type of wave shape can be used.

### EXAMPLES

Hereinafter, the present invention will be described in more detail on the basis of synthesis examples and examples.

Structural formulae of Intermediates 1 and 2 to be produced in Synthesis Examples 1 and 2 are as shown below.

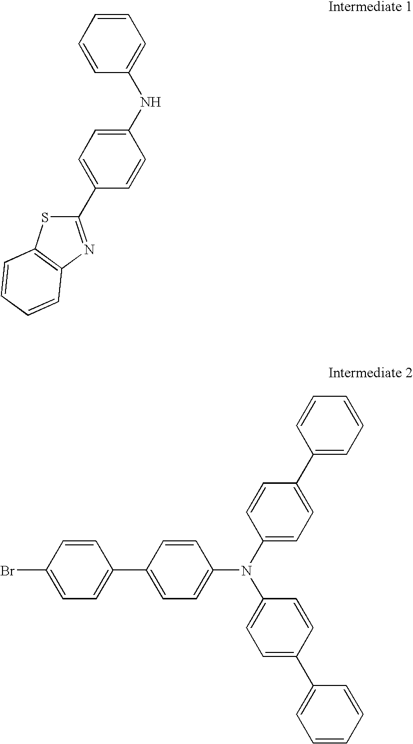

### Synthesis Example 1

### Synthesis of Intermediate 1

In a stream of argon, 5.5 g of aniline, 14.5 g of 2-(4-bromophenyl)benzothiazole, 6.8 g of sodium t-butoxide (manufactured by HIROSHIMA WAKO CO., LTD.), 0.46 g of tris(dibenzylideneacetone)dipalladium(0) (manufactured by Aldrich), and 300 mL of anhydrous toluene were loaded, and the whole was subjected to a reaction at 80° C. for 8 hours.

After the resultant had been cooled, 500 mL of water were added to the resultant, and the mixture was subjected to cerite filtration. The filtrate was extracted with toluene and dried with anhydrous magnesium sulfate. The dried product was concentrated under reduced pressure, and the resultant coarse product was subjected to column purification and recrystallized with toluene. The crystal was taken by filtration, and was then dried, whereby 10.8 g of a pale yellow powder were obtained. The powder was identified as Intermediate 1 by FD-MS analysis.

### Synthesis Example 2

### Synthesis of Intermediate 2

20.0 g of 4-bromobiphenyl (manufactured by TOKYO CHEMICAL INDUSTRY CO., LTD.), 8.64 g of sodium t-butoxide (manufactured by Wako Pure Chemical Industries, Ltd.), and 84 mg of palladium acetate (manufactured by Wako Pure Chemical Industries, Ltd.) were loaded into a 200-mL three-necked flask. Further, a stirring rod was placed in the flask, and rubber caps were set on both side ports of the flask. A condenser for reflux was inserted into the central port of the flask, and a three-way cock and a balloon in which an argon gas was sealed were set above the condenser. The inside of the system was replaced with the argon gas in the balloon three times by using a vacuum pump.

Next, 120 mL of anhydrous toluene (manufactured by HIROSHIMA WAKO CO., LTD.), 4.08 mL of benzylamine (manufactured by TOKYO CHEMICAL INDUSTRY CO., LTD.), and 338 μL of tris-t-butylphosphine (manufactured by SIGMA-ALDRICH, 2.22-mol/L toluene solution) were added to the flask by using a syringe through a rubber septum, and the whole was stirred for 5 minutes at room temperature. Next, the flask was set in an oil bath, and was gradually heated to 120° C. while the solution was stirred. 7 hours after that, the flask was lifted off the oil bath so that the reaction would be completed. The flask was left under an argon atmosphere for 12 hours. The reaction solution was transferred to a separating funnel, and 600 mL of dichloromethane were added to dissolve the precipitate. After the resultant had been washed with 120 mL of a saturated brine, an organic layer was dried with anhydrous potassium carbonate. The solvent of the organic layer obtained by separating potassium carbonate by filtration was removed by distillation. 400 mL of toluene and 80 mL of ethanol were added to the resultant residue. A drying tube was attached and the resultant was heated to 80° C. so that the residue would be completely dissolved. After that, the resultant was left for 12 hours, and was slowly cooled to room temperature for recrystallization. The precipitated crystal was separated by filtration, and was dried in a vacuum at 60° C., whereby 13.5 g of N,N-di-(4-biphenylyl)benzylamine were obtained.

1.35 g of N,N-di-(4-biphenylyl)benzylamine and 135 mg of palladium-activated carbon (manufactured by HIROSHIMA WAKO CO., LTD., palladium content 10 wt %) were loaded into a 300-mL one-necked flask, and 100 mL of chloroform and 20 mL of ethanol were added to dissolve the mixture. Next, a stirring rod was placed in the flask. After that, a three-way cock mounted with a balloon filled with 2 L of a hydrogen gas was attached to the flask, and the inside of the flask system was replaced with the hydrogen gas ten times by using a vacuum pump. The balloon was newly filled with a hydrogen gas in an amount corresponding to the reduced amount so that the volume of the hydrogen gas would be 2 L again. After that, the solution was vigorously stirred at room temperature for 30 hours. After that, 100 mL of dichloromethane were added to the resultant, and the catalyst was separated by filtration. Next, the resultant solution was transferred to a separating funnel, and was washed with 50 mL of a saturated aqueous solution of sodium hydrogen carbonate. After that, an organic layer was separated and dried with anhydrous potassium carbonate. After the resultant had been filtrated, the solvent was removed by distillation, and 50 mL of toluene were added to the resultant residue for recrystallization. The precipitated crystal was separated by filtration, and was dried in a vacuum at 50° C., whereby 0.99 g of di-4-biphenylylamine was obtained.

In a stream of argon, 10 g of di-4-biphenylylamine, 9.7 g of 4,4-dibromobiphenyl (manufactured by TOKYO CHEMICAL INDUSTRY CO., LTD.), 3 g of sodium t-butoxide (manufactured by HIROSHIMA WAKO CO., LTD.), 0.5 g of bis(triphenylphosphine)palladium(II) chloride (manufactured by TOKYO CHEMICAL INDUSTRY CO., LTD.), and 500 mL of xylene were loaded, and the whole was subjected to a reaction at 130° C. for 24 hours. After the resultant had been cooled, 1,000 mL of water were added to the resultant, and the mixture was subjected to celite filtration. The filtrate was extracted with toluene and dried with anhydrous magnesium sulfate. The dried product was concentrated under reduced pressure, and the resultant crude product was subjected to column purification. The purified product was recrystallized with toluene. The crystal was taken by filtration, and was then dried, whereby 9.1 g of Intermediate 2 (4-bromo-N,N-dibiphenylyl-4-amino-1,1′-biphenyl) were obtained.

Structural formulae of Compounds H1 and H2 to be produced in Examples of Synthesis 1 and 2 and each serving as the aromatic amine derivative of the present invention are as shown below.

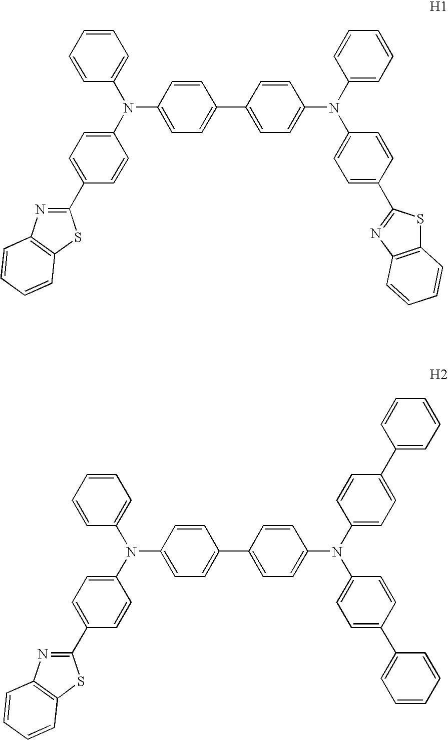

### Example of Synthesis 1

### Synthesis of Compound H1

In a stream of argon, 3.4 g of N,N′-diphenylbenzidine, 6.1 g of 2-(4-bromophenyl)benzothiazole, 2.6 g of sodium t-butoxide (manufactured by HIROSHIMA WAKO CO., LTD.), 92 mg of tris(dibenzylideneacetone)dipalladium(0) (manufactured by Aldrich), 42 mg of tri-t-butylphosphine, and 100 mL of anhydrous toluene were loaded, and the whole was subjected to a reaction at 80° C. for 8 hours.

After the resultant had been cooled, 500 mL of water were added to the resultant, and the mixture was subjected to cerite filtration. The filtrate was extracted with toluene and dried with anhydrous magnesium sulfate. The dried product was concentrated under reduced pressure, and the resultant coarse product was subjected to column purification and recrystallized with toluene. The crystal was taken by filtration, and was then dried, whereby 4.0 g of a pale yellow powder were obtained. The powder was identified as Compound H1 by FD-MS (field desorption mass spectrometry) analysis.

### Example of Synthesis 2

### Synthesis of Compound H2

In a stream of argon, 6.1 g of Intermediate 1, 11.0 g of Intermediate 2, 2.6 g of sodium t-butoxide (manufactured by HIROSHIMA WAKO CO., LTD.), 92 mg of tris(dibenzylideneacetone)dipalladium(0) (manufactured by Aldrich), 42 mg of tri-t-butylphosphine, and 100 mL of anhydrous toluene were loaded, and the whole was subjected to a reaction at 80° C. for 8 hours.

After having been cooled, the resultant was added with 500 ml of water, and the mixture was subjected to celite filtration. The filtrate was extracted with toluene and dried with anhydrous magnesium sulfate. The resultant was concentrated under reduced pressure, and the resultant crude product was subjected to column purification. Then, the resultant was recrystallized with toluene, and the recrystallized product was separated by filtration and dried, thereby yielding 12.2 g of pale yellow powder. The powder was identified as Compound H2 by FD-MS (field desorption mass spectrometry) analysis.

### Example 1

### Production of Organic EL Device

A glass substrate with an ITO transparent electrode measuring 25 mm wide by 75 mm long by 1.1 mm thick (manufactured by GEOMATEC Co., Ltd.) was subjected to ultrasonic cleaning in isopropyl alcohol for 5 minutes. After that, the substrate was subjected to UV ozone cleaning for 30 minutes.

The glass substrate with the transparent electrode line after the washing was mounted on a substrate holder of a vacuum deposition device. First, Compound H1 described above was formed into a film having a thickness of 60 nm on the surface on the side where the transparent electrode line was formed to cover the transparent electrode. The H1 film functions as a hole injecting layer. Compound layer of TBDB described below was formed into a film having a thickness of 20 nm on the H1 film. The film functions as a hole transporting layer. Further, Compound EM1 to be described below was deposited from the vapor and formed in to a film having a thickness of 40 nm. Simultaneously with this formation, Amine Compound D1 having a styryl group to be described below, as a light emitting molecule, was deposited from the vapor in such a manner that a weight ratio of EM1 to D1 would be 40:2. The film functions as a light emitting layer.

Alq to be described below was formed into a film having a thickness of 10 nm on the resultant film. The film functions as an electron injecting layer. After that, Li serving as a reducing dopant (Li source: manufactured by SAES Getters) and Alq were subjected to co-deposition. Thus, an Alq:Li film (having a thickness of 10 nm) was formed as an electron injecting layer (cathode). Metal Al was deposited from the vapor onto the Alq:Li film to form a metal cathode. Thus, an organic EL device was formed.

In addition, the current efficiency of the resultant organic EL device was measured, and the luminescent color of the device was observed. A current efficiency at 10 mA/cm² was calculated by measuring a luminance by using a CS1000 manufactured by Minolta. Further, the half lifetime of light emission in DC constant current driving at an initial luminance of 5,000 cd/m² and room temperature was measured. Table 1 shows the results thereof.

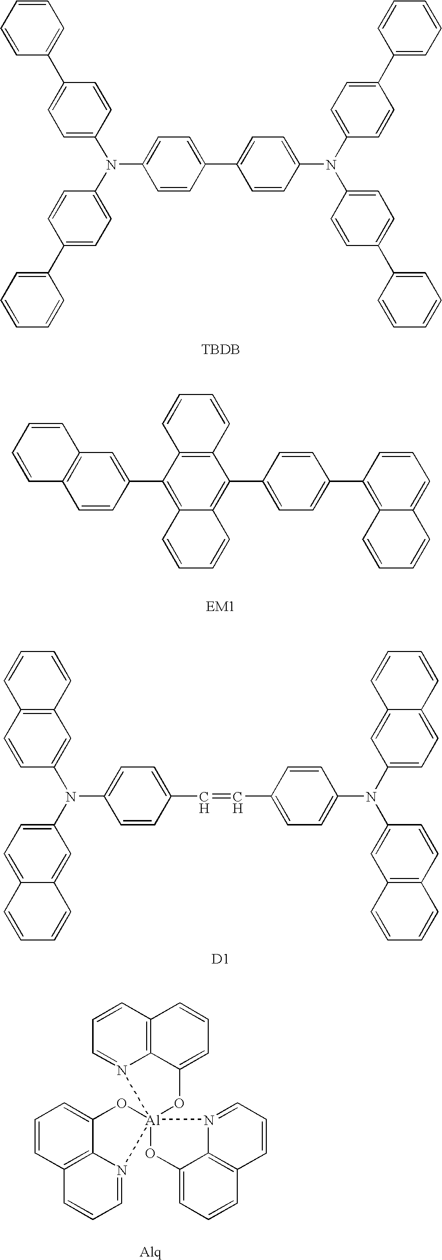

### Example 2

### Production of Organic EL Device

An organic EL device was produced in the same manner as in Example 1 except that: HB1 was used as a material for a hole injecting layer instead of H1; and H1 was used as a hole transporting layer instead of TBDB.

The current efficiency of the resultant organic EL device was measured, and the luminescent color of the device was observed. Further, the half lifetime of light emission in DC constant current driving at an initial luminance of 5,000 cd/m² and room temperature was measured. Table 1 shows the results.

### Example 3

### Production of Organic EL Device

An organic EL device was produced in the same manner as in Example 1 except that H2 was used as a hole injecting layer instead of H1.

The current efficiency of the resultant organic EL device was measured, and the luminescent color of the device was observed Further, the half lifetime of light emission in DC constant current driving at an initial luminance of 5,000 cd/m² and room temperature was measured. Table 1 shows the results.

### Comparative Example 1

An organic EL device was produced in the same manner as in Example 1 except that HB1 was used as a hole transporting and injecting layer instead of Compound H1.

The current efficiency of the resultant organic EL device was measured, and the luminescent color of the device was observed. Further, the half lifetime of light emission in DC constant current driving at an initial luminance of 5,000 cd/m² and room temperature was measured. Table 1 shows the results.

TABLE 1
Results of evaluation of devices

| | Hole | Hole | | | Half |
|---|---|---|---|---|---|
| | injectin | transporting | Voltage | Luminescent | lifetime |
| Example | g layer | layer | (V) | color | (h) |
| 1 | H1 | TBDB | 6.1 | Blue | 430 |
| 2 | HB1 | H1 | 6.5 | Blue | 350 |
| 3 | H2 | TBDB | 6.3 | Blue | 410 |
| Comparative | HB1 | TBDB | 7.1 | Blue | 280 |
| example 1 | | | | | |

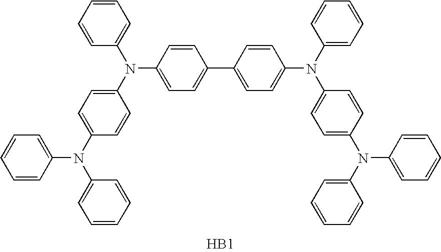

### INDUSTRIAL APPLICABILITY

As described above in detail, the aromatic amine derivative of the present invention reduces the driving voltage. In addition, a molecule of the derivative hardly crystallizes. The incorporation of the derivative into an organic thin film layer can: improve the yield in which an organic EL device is produced; and realize an organic EL device having a long lifetime.

1. An aromatic amine derivative represented by the following general formula (1): 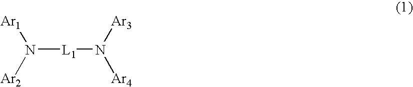
where:
L₁ represents a substituted or unsubstituted arylene group having 5 to 50 ring carbon atoms, or a substituted or unsubstituted heteroarylene group having 5 to 50 ring carbon atoms; and
at least one of Ar₁ to Ar₄ is represented by the following general formula (2) 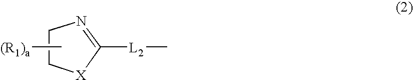
where
R₁ represents a hydrogen atom, a substituted or unsubstituted aryl group having 5 to 50 ring carbon atoms, a substituted or unsubstituted alkyl group having 1 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, an amino group substituted by a substituted or unsubstituted aryl group having 5 to 50 ring carbon atoms, a halogen atom, a cyano group, a nitro group, a hydroxy group, or a carboxyl group,
a represents an integer of 0 to 2,
X represents a sulfur atom, an oxygen atom, a selenium atom, or a tellurium atom,
L₂ represents a substituted or unsubstituted arylene group having 5 to 50 ring carbon atoms, or a substituted or unsubstituted heteroarylene group having 5 to 50 ring carbon atoms, and
multiple R₁s may be bonded to each other to form a saturated or unsaturated, five- or six-membered cyclic structure which may be substituted; and
remaining groups of Ar₁ to Ar₄ none of which is represented by the general formula (2) each independently represent a substituted or unsubstituted aryl group having 5 to 50 ring carbon atoms, or a substituted or unsubstituted heteroaryl group having 5 to 50 ring carbon atoms.

2. An aromatic amine derivative according to claim 1, wherein Ar₁ in the general formula (1) is represented by the general formula (2).

3. An aromatic amine derivative according to claim 1, wherein Ar₁ and Ar₂ in the general formula (1) are each represented by the general formula (2).

4. An aromatic amine derivative according to claim 1, wherein Ar₁ and Ar₃ in the general formula (1) are each represented by the general formula (2).

5. An aromatic amine derivative according to claim 1, wherein three or more of Ar₁ to Ar₄ in the general formula (1) are different from one another and wherein the aromatic amine compound is asymmetric.

6. An aromatic amine derivative according to claim 1, wherein three of Ar₁ to Ar₄ in the general formula (1) are identical to one another and wherein the aromatic amine compound is asymmetric.

7. An aromatic amine derivative according to any one of claims 1 to 6, wherein L₁ in the general formula (1) represents a biphenylene group, a terphenylene group, or a fluorenylene group.

8. An aromatic amine derivative according to any one of claims 1 to 7, wherein L₂ in the general formula (2) represents a phenylene group or a naphthylene group.

9. An aromatic amine derivative according to claim 1, wherein at least one of Ar₁ to Ar₄ in the general formula (1) is represented by the following general formula (3): 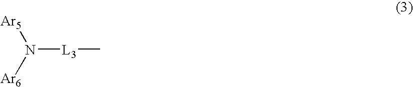
where:
Ar₅ and Ar₆ each independently represent a substituted or unsubstituted aryl group having 5 to 50 ring carbon atoms, a substituted or unsubstituted heteroaryl group having 5 to 50 ring carbon atoms, or a substituent represented by the general formula (2); and
L₃ represents a substituted or unsubstituted arylene group having 5 to 50 ring carbon atoms, or a substituted or unsubstituted heteroarylene group having 5 to 50 ring carbon atoms.

10. An aromatic amine derivative according to claim 1, wherein Ar₂ in the general formula (1) is represented by the general formula (3).

11. An aromatic amine derivative according to claim 1, wherein Ar₂ and Ar₄ in the general formula (1) are each independently represented by the general formula (3).

12. An aromatic amine derivative according to any one of claims 1 to 11, wherein X in the general formula (2) represents a sulfur atom.

13. An aromatic amine derivative according to any one of claims 1 to 12, wherein it is a material for an organic electroluminescence device.

14. An aromatic amine derivative according to any one of claims 1 to 12, wherein it is a hole transporting material for an organic electroluminescence device.

15. An organic electroluminescence device, comprising an organic thin film layer composed of one or more layers including at least a light emitting layer, the organic thin film layer being interposed between a cathode and an anode, wherein at least one layer of the organic thin film layer contains the aromatic amine derivative according to any one of claims 1 to 12 alone or as a component of a mixture.

16. An organic electroluminescence device according to claim 15, wherein the organic thin film layer has a hole transporting layer, and the aromatic amine derivative according to any one of claims 1 to 12 is incorporated into the hole transporting layer.

17. An organic electroluminescence device according to claim 15, wherein the organic thin film layer has a hole injecting layer, and the aromatic amine derivative according to any one of claims 1 to 12 is incorporated into the hole injecting layer.

18. An organic electroluminescence device according to claim 15, wherein the aromatic amine derivative according to any one of claims 1 to 12 is incorporated as a main component into a hole injecting layer.
